(19) United States
(12) Patent Application Publication
(10) Pub. No.: US 2024/0382498 A1
(43) Pub. Date: Nov. 21, 2024

(54) NOVEL INHIBITOR OF HYPE-MEDIATED AMPYLATION

(71) Applicant: Purdue Research Foundation, West Lafayette, IN (US)

(72) Inventor: Seema Mattoo, West Lafayette, IN (US)

(21) Appl. No.: 18/664,841

(22) Filed: May 15, 2024

Related U.S. Application Data
(60) Provisional application No. 63/466,935, filed on May 16, 2023.

(51) Int. Cl. A61K 31/549; A61K 31/4025; A61K 31/4184; A61K 31/4245; A61K 31/433; A61K 31/437; A61K 31/443; A61K 31/4439; A61K 31/4709; A61K 31/506; C07D 207/34; C07D 401/06; C07D 401/14; C07D 403/04; C07D 405/12; C07D 413/04; C07D 413/14; C07D 417/12; C07D 471/04; C07D 513/10

(52) U.S. Cl. CPC A61K 31/549; A61K 31/4025; A61K 31/4184; A61K 31/4245; A61K 31/433; A61K 31/437; A61K 31/443; A61K 31/4439; A61K 31/4709; A61K 31/506; C07D 207/34; C07D 401/06; C07D 401/14; C07D 403/04; C07D 405/12; C07D 413/04; C07D 413/14; C07D 417/12; C07D 471/04; C07D 513/10

(57) ABSTRACT

The present application provides for compounds, pharmaceutical formulations and methods for the treatment of HYPE mediated disease. The present application generally relates to a method to treat HYPE-mediated AMPylation associated disease in a patient. The present application provides for a method of optimized high-throughput screening for compounds which are effective for modulating HYPE-mediated AMPylation. The present application particularly provides for pharmaceutical formulations, compounds and methods of use.

### CROSS-REFERENCE TO RELATED APPLICATION

This Application relates to and claims the benefit of U.S. provisional application 63/466,935, filed on May 16, 2023. The contents of which are expressly incorporated herein by reference in its entirety into this present disclosure.

### GOVERNMENT SUPPORT CLAUSE

This invention was made with government support under grant GM100092 awarded by the National Institutes of Health. The government has certain rights in the invention.

### TECHNICAL FIELD

The present application relates to an optimized high-throughput screening (HTS) method for discovery of activators or inhibitors of HYPE-mediated AMPylation. The present application relates generally to compounds and their use for inhibiting, moderating, and/or regulating HYPE-mediated AMPylation. Thus the present disclosure provides for the novel compounds directed against HYPE-mediated AMPylation. In a particular embodiment the disclosure provides for novel pharmaceutical formulations and compositions for treating HYPE mediated disease, and for pharmaceutical products for treating such. Another aspect of the present disclosure relates to methods for moderating, treating, and/or ameliorating HYPE-mediated AMPylation related pathologies, especially as found in relation to neurodegeneration and other neurological diseases.

### BACKGROUND AND BRIEF SUMMARY

This section introduces aspects that may help facilitate a better understanding of the disclosure. Accordingly, these statements are to be read in this light and are not to be understood as admissions about what is or is not prior art.

Fic (filamentation induced by cyclic AMP) are an evolutionarily conserved family of enzymes. While they catalyze a wide range of posttranslational modifications to modulate cell signaling, most Fics studied to date carry out AMPylation (adenyl(yl)ation)(Yarbrough et al 2009; Worby et al. 2009). This entails the covalent attachment of an AMP from ATP unto target protein substrates at a serine, threonine, or tyrosine. AMPylation is enabled by the Fic motif—HXFX(D/E)(G/A)N(G/K)RXXR—within the structurally conserved active site of the Fic domain. The leading Fic motif histidine initiates AMPylation by deprotonating the target hydroxyl group, followed by a nucleophilic attack of the activated oxygen on the alpha phosphate of ATP. The reaction is completed by electron shuttling to the pyrophosphate leaving group.

Humans contain a single Fic protein HYPE (Huntington Yeast Interacting Partner E) or FICD. HYPE resides in the ER (endoplasmic reticulum) where it AMPylates the heat shock chaperone BiP (Binding Immunoglobulin Protein) (Ham et al. 2014; Sanyal et al. 2015). AMPylation of BiP renders it an inactive chaperone during homeostasis. However, in response to accumulation of misfolded proteins in the ER, BiP is deAMPylated to become an active chaperone (Ham et al. 2014; Preissler et al., 2017). In addition to BiP, we recently identified alpha synuclein as a target of HYPE AMPylation (Sanyal et al. 2019). We showed that AMPylated alpha synuclein saw a reduction of several cytotoxic phenotypes associated with Parkinson's disease such as fibrillation and membrane permeability. Interestingly, manipulation of BiP AMPylation was also shown to indirectly influence the aggregative state of alpha synuclein and other disease-linked proteins (i.e. β-amyloid in Alzheimer's disease and PolyQ in Huntingtin's disease) (Truttmann et al. 2018).

Following the discoveries of BiP and alpha synuclein, a wave of putative substrates for HYPE AMPylation are being revealed (Kielkowski et al. 2020). This HYPE AMPylome encompasses proteins involved in disease pathways ranging from cancer to diabetes to neurodegeneration. Given HYPE's central cellular role, we wanted to target it for broad therapeutic intervention. We, therefore, conducted a fluorescence polarization (FP)-based, dual pilot high throughput screen (HTS) to discover activators and inhibitors of HYPE-mediated AMPylation (Camara et al. 2020). After screening nearly 10,000 compounds, we found that calmidazolium and aurintricarboxylic acid can reliably enhance and inhibit AMPylation in vitro, respectively. Despite these bioactivities, the ultimate therapeutic utility of these compounds is limited by their multiple cellular targets. Calmidazolium, for example, interacts with no less than five other proteins, including its namesake the ER calcium-binding resident calmodulin (Camara et al. 2020; Lewallen et al. 2015; Gietzen et al. 1981; Sunagawa et al. 2000; James et al. 2009). Likewise, aurintricarboxylic acid interferes ribonuclease and topoisomerase II, among others (Camara et al. 2020; McCune et al. 1989; Hashem et al. 2009; Obrecht et al., 2019). The promiscuous nature of these hits is not surprising given their origination from a pilot screen library (LOPAC1280) containing pharmacologically active compounds (Coan et al. 2009). Nevertheless, we wanted to conduct a screen containing novel, HYPE-specific molecules. We expanded this screen to over 30,000 compounds for activators, and inhibitors, and included a central nervous system (CNS)-compatible library to account for HYPE's role in neurodegeneration.

The present disclosure provides for methods for treating neurodegenerative diseases which are impacted by HYPE-mediated AMPylation.

The present disclosure provides for pharmaceutical compositions for treating neurodegenerative diseases which are impacted by HYPE-mediated AMPylation. Thus the present disclosure provides for compounds and pharmaceutical compositions comprising such compounds for treating neurodegenerative disease. In one aspect the disclosure provides for treating neurodegeneration as a consequence of aging. In another aspect the disclosure specifically provides for treatment of diseases such as Alzheimer's Disease, Huntington's Disease and Parkinson's Disease.

The present disclosure provides for compounds which are useful for moderating, altering, inhibiting, and/or interfering with HYPE-mediated AMPylation.

The present disclosure provides for methods for screening for such compounds.

The present disclosure provides for a pharmaceutical formulation comprising a compound having a formula (I)

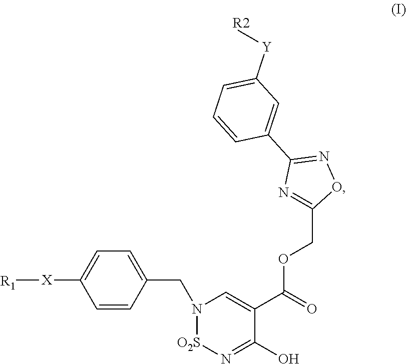

or a pharmaceutically acceptable salt thereof, wherein
X is H or O;
Y is H or O;
R₁ is a C₁-C₁₂ alkyl;
R₂ is a C₁-C₁₂ alkyl; heteroalkyl, heteroalkenyl, heteroalkynyl, cycloalkyl, heterocyloalkyl, cycloalkenyl, heterocycloakenyl, heterocyclyl, or cycloalkene,
and a pharmaceutically acceptable carrier, excipient or diluent.

The present disclosure provides for a formulation as above wherein the compound is selected from the group consisting of:

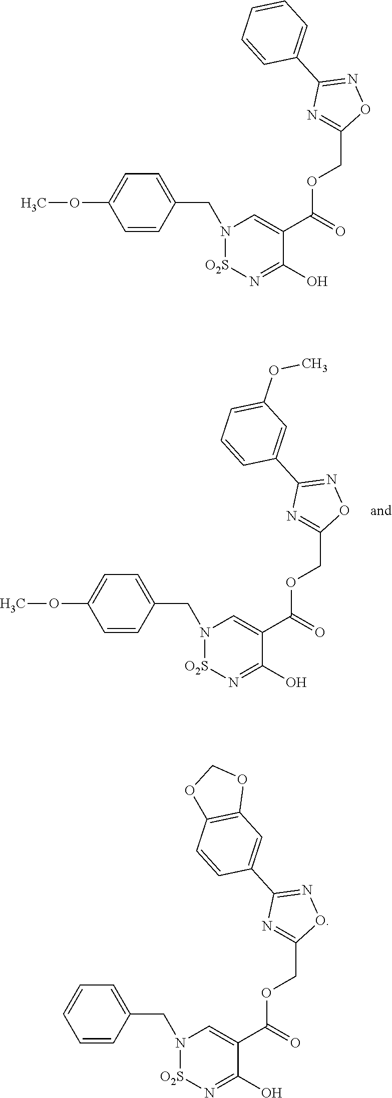 [SMILES: COc1ccc(CN2C=C(C(=O)OCc3nc(-c4ccccc4)no3)C(O)=NS2(=O)=O)cc1 ; COc1ccc(CN2C=C(C(=O)OCc3nc(-c4cccc(OC)c4)no3)C(O)=NS2(=O)=O)cc1 ; "and" ; O=C(OCc1nc(-c2ccc3c(c2)OCO3)no1)C1=CN(Cc2ccccc2)S(=O)(=O)N=C1O]

The present disclosure provides for a pharmaceutical composition comprising a compound having a formula (II)

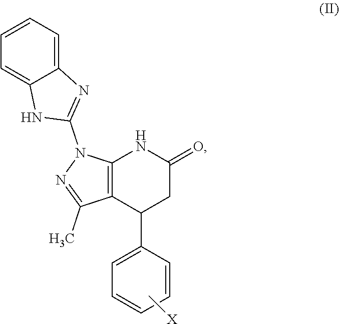

or a pharmaceutically acceptable salt thereof, wherein
X is 1 or 2 optionally bound halo-substituent to the ring structure.

The present disclosure provides for a formulation as above selected from the group consisting of

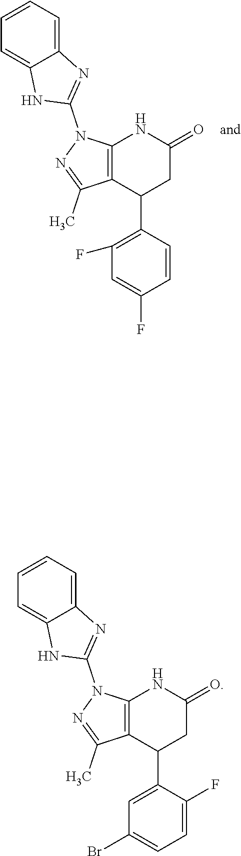 [SMILES: Cc1nn(-c2nc3ccccc3[nH]2)c2c1C(c1ccc(F)cc1F)CC(=O)N2 ; "and" ; Cc1nn(-c2nc3ccccc3[nH]2)c2c1C(c1cc(Br)ccc1F)CC(=O)N2]

The present disclosure provides for a pharmaceutical formulation comprising a compound having a formula (III)

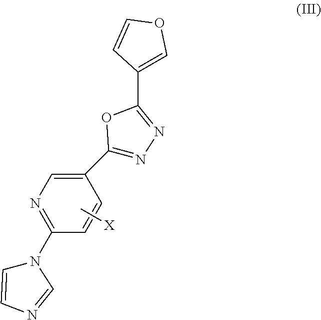

or a pharmaceutically acceptable salt thereof, wherein
X is 1 or 2 optionally bound halo-substituent to the ring structure.

The present disclosure provides for a formulation as above having the formula

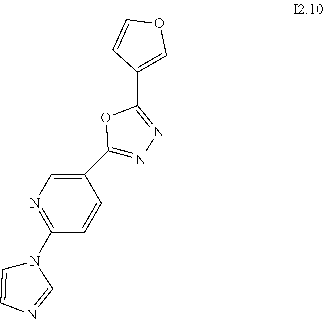 [SMILES: c1cn(-c2ccc(-c3nnc(-c4ccoc4)o3)cn2)cn1]

The present disclosure provides for a method for treating a patient for HYPE-mediated neurodegenerative disease comprising administering a therapeutically effective amount of one or more of the formulations above to the patient in need of relief from said disease.

A pharmaceutical composition for the treatment of HYPE-mediated neurodegenerative disease comprising therapeutically effective amount of one or more of a formulation as above, together with one or more anti-infective agents and one or more pharmaceutically acceptable carriers, diluents, and excipients.

The present disclosure provides for a compound selected from the group consisting of:

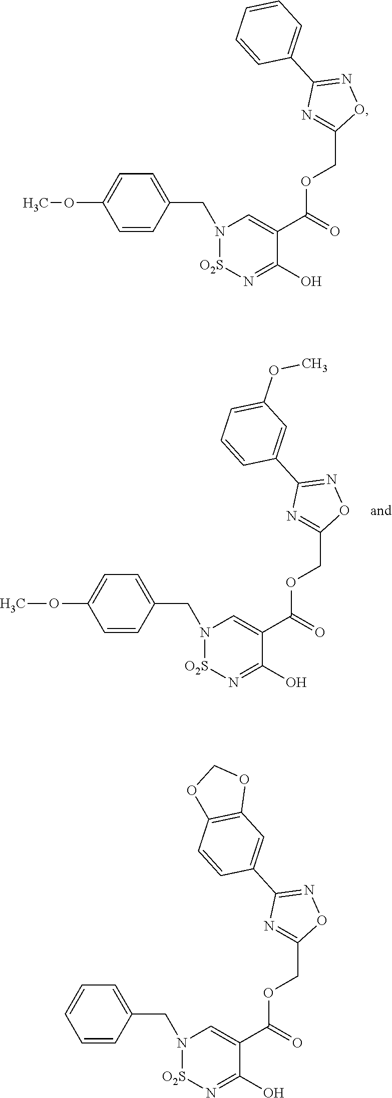 [SMILES: COc1ccc(CN2C=C(C(=O)OCc3nc(-c4ccccc4)no3)C(O)=NS2(=O)=O)cc1 ; COc1ccc(CN2C=C(C(=O)OCc3nc(-c4cccc(OC)c4)no3)C(O)=NS2(=O)=O)cc1 ; "and" ; O=C(OCc1nc(-c2ccc3c(c2)OCO3)no1)C1=CN(Cc2ccccc2)S(=O)(=O)N=C1O]

which is an activator of HYPE-mediated AMPylation.

The present disclosure provides for a compound selected from the group consisting of:

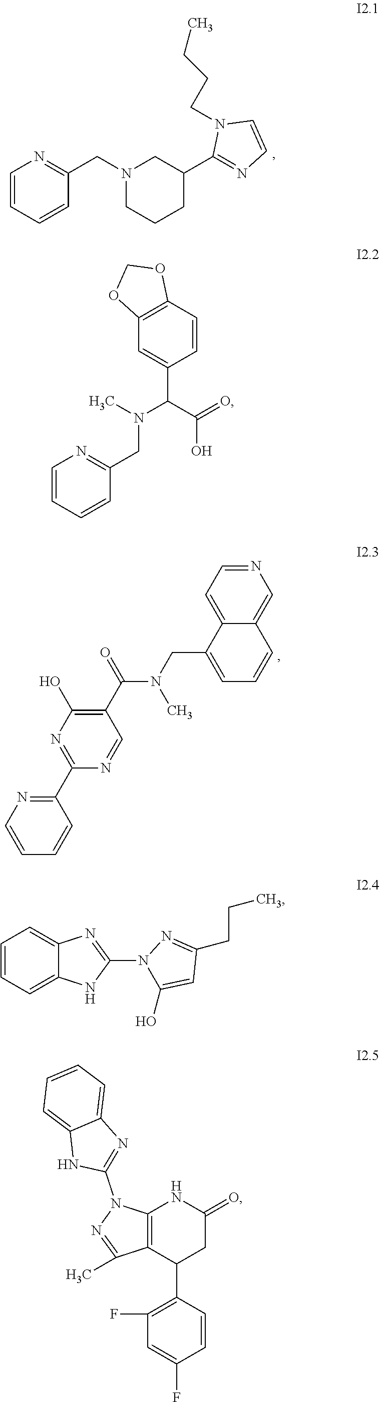

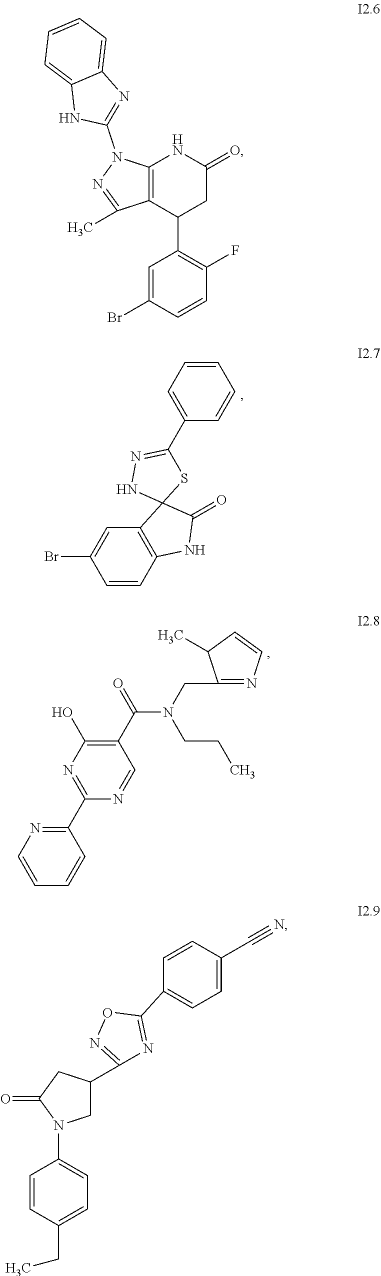

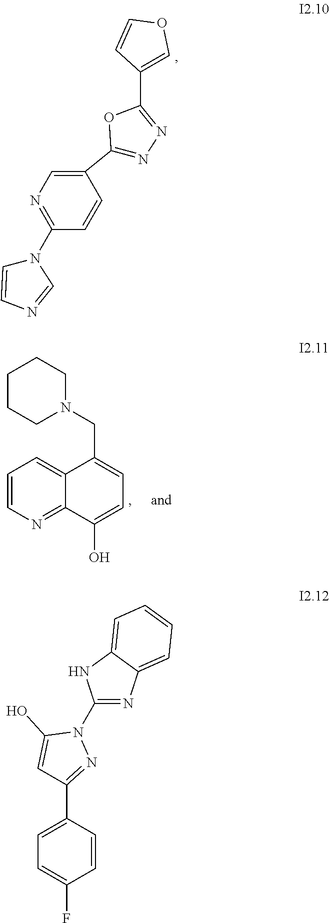

which is an inhibitor of HYPE-mediated AMPylation.

The present disclosure provides for a pharmaceutical composition comprising a therapeutically effective amount of a compound of as described above and a pharmaceutically acceptable carrier, excipient or diluent.

The present disclosure provides for a pharmaceutical composition comprising a therapeutically effective amount of a compound as described above and a pharmaceutically acceptable carrier, excipient or diluent.

The present disclosure provides for a method for treating a patient for HYPE-mediated neurodegenerative disease comprising administering a therapeutically effective amount of the compound of claim 12 to the patient in need of relief from said disease.

The present disclosure provides for a method for treating a patient for HYPE-mediated neurodegenerative disease comprising administering a therapeutically effective amount of the compound of claim 13 to the patient in need of relief from said disease.

The present disclosure provides for a method for screening for an activator of HYPE-mediated AMPylation comprising preparing a test environment having a modified AMPylase HYPE in a Mg buffer, contacting a test small molecule with the test environment, and detecting signal, where said method is amenable to tracking HYPE's enzymatic activity at scale with automation.

The present disclosure provides for a method as above using hypoactive AMPylase WT HYPE and Mg Buffer to screen for potential specific activators.

The present disclosure provides for a method as above using hyperactive E234G HYPE and Mg Buffer to screen for potential specific inhibitors.

The present disclosure provides for a compound having a formula (I)

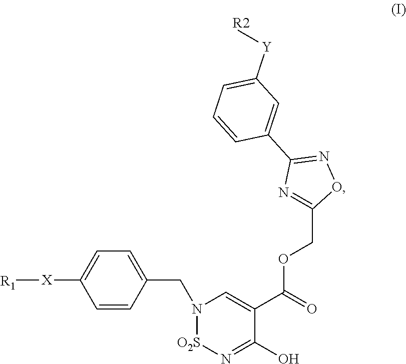

or a pharmaceutically acceptable salt thereof, wherein
X is H or O;
Y is H or O;
R₁ is a C₁-C₁₂ alkyl;
R₂ is a C₁-C₁₂ alkyl; heteroalkyl, heteroalkenyl, heteroalkynyl, cycloalkyl, heterocyloalkyl, cycloalkenyl, heterocycloakenyl, heterocyclyl, or cycloalkene.

The present disclosure provides for a compound as in formula I, wherein the compound is selected from the group consisting of:

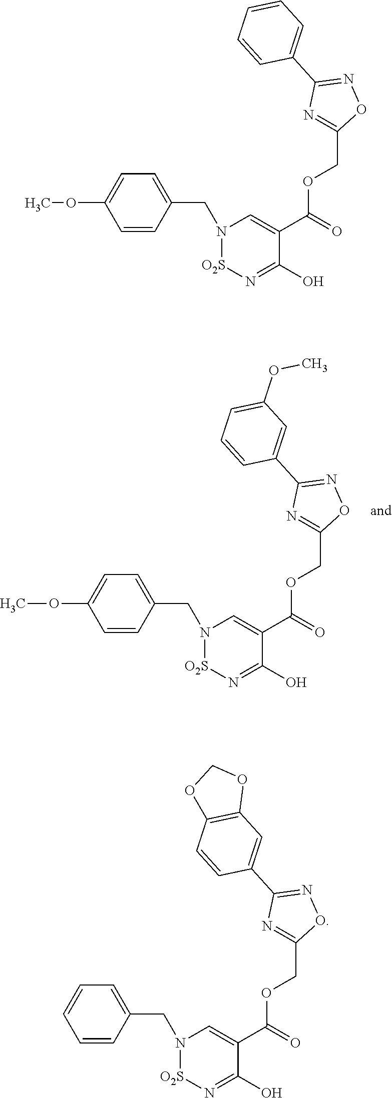 [SMILES: COc1ccc(CN2C=C(C(=O)OCc3nc(-c4ccccc4)no3)C(O)=NS2(=O)=O)cc1 ; COc1ccc(CN2C=C(C(=O)OCc3nc(-c4cccc(OC)c4)no3)C(O)=NS2(=O)=O)cc1 ; "and" ; O=C(OCc1nc(-c2ccc3c(c2)OCO3)no1)C1=CN(Cc2ccccc2)S(=O)(=O)N=C1O]

The present disclosure provides for a compound of formula (II)

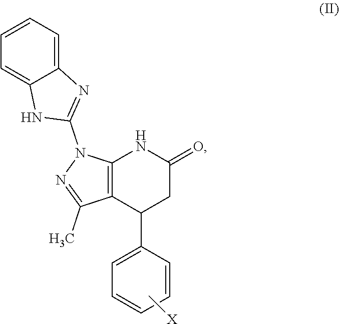

or a pharmaceutically acceptable salt thereof, wherein
X is 1 or 2 optionally bound halo-substituent to the ring structure.

The present disclosure provides for a compound of formula II selected from the group consisting of

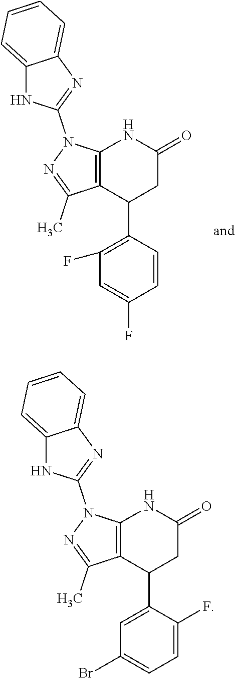 [SMILES: Cc1nn(-c2nc3ccccc3[nH]2)c2c1C(c1ccc(F)cc1F)CC(=O)N2 ; Cc1nn(-c2nc3ccccc3[nH]2)c2c1C(c1cc(Br)ccc1F)CC(=O)N2]

The present disclosure provides for a compound of formula III

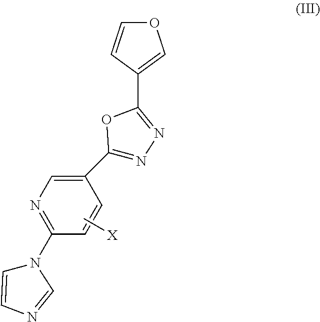

or a pharmaceutically acceptable salt thereof, wherein
X is 1 or 2 optionally bound halo-substituent to the ring structure.

The present disclosure provides for a compound of formula III which is

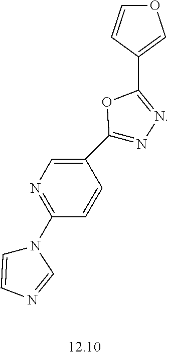 [SMILES: c1cn(-c2ccc(-c3nnc(-c4ccoc4)o3)cn2)cn1]

The present disclosure provides for a method for treating a patient for HYPE-mediated neurodegenerative disease comprising administering a therapeutically effective amount of the compound of formula I, II, or III to the patient in need of relief from said disease, or such compound as shown in FIG. 4 and FIG. 9.

In the present disclosure, treatment of a neurogenerative disease is provided by an agonist or antagonist of HYPE-mediated AMPylase activity. In particular, the treatment of the present disclosure may be applied to patients suffering from Alzheimer's disease, Huntington's disease and the like.

The present disclosure provides for pharmaceutical compositions for the treatment of HYPE-mediated neurodegenerative disease comprising therapeutically effective amount of a compound of the present disclosure, together with one or more agents for treating neurodegenerative disease conditions, and one or more pharmaceutically acceptable carriers, diluents, and excipients.

The present disclosure provides for a compound which is an activator of HYPE-mediated AMPylation selected from the group consisting of:

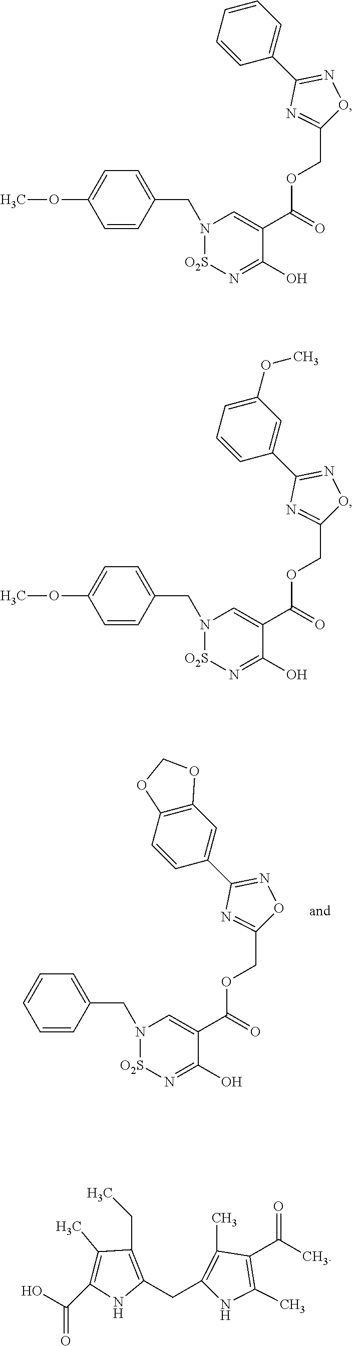 [SMILES: COc1ccc(CN2C=C(C(=O)OCc3nc(-c4ccccc4)no3)C(O)=NS2(=O)=O)cc1 ; COc1ccc(CN2C=C(C(=O)OCc3nc(-c4cccc(OC)c4)no3)C(O)=NS2(=O)=O)cc1 ; O=C(OCc1nc(-c2ccc3c(c2)OCO3)no1)C1=CN(Cc2ccccc2)S(=O)(=O)N=C1O ; CCc1c(Cc2[nH]c(C)c(C(C)=O)c2C)[nH]c(C(=O)O)c1C]

The present disclosure provides for a compound which is an inhibitor of HYPE-mediated AMPylation selected from the group consisting of:

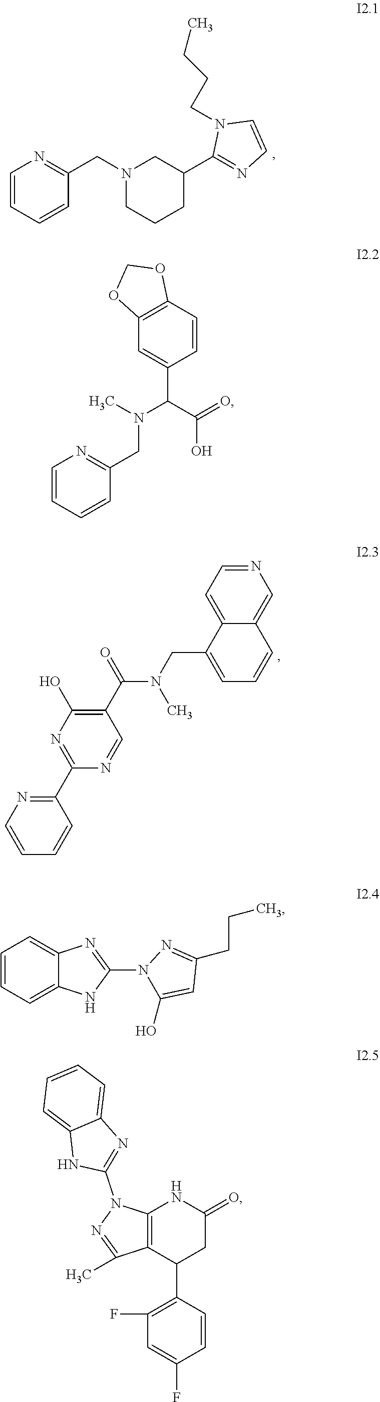

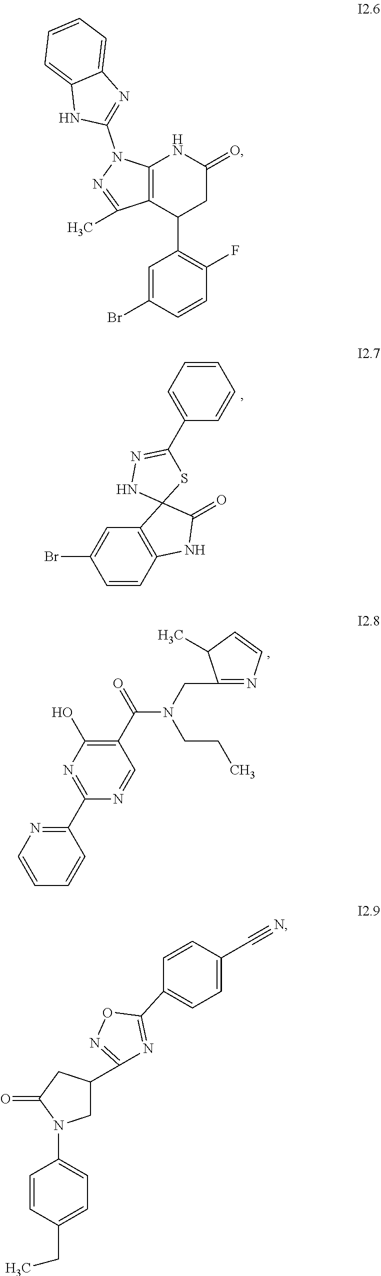

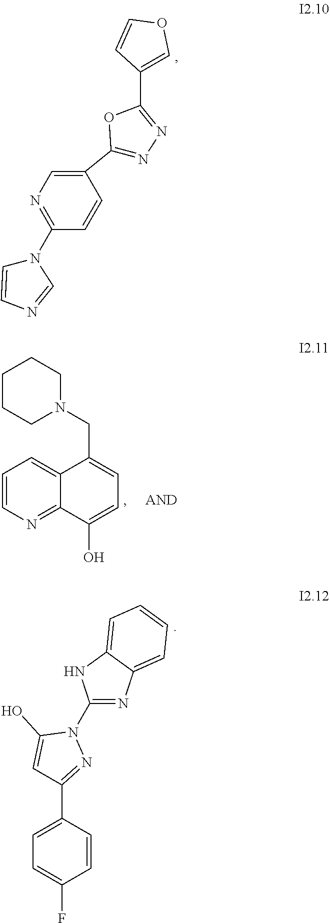

### BRIEF DESCRIPTION OF THE DRAWINGS

The above and other objects, features, and advantages of the present disclosure will become more apparent when taken in conjunction with the following drawings, and wherein:

FIG. 1 (FIG. 1A-C) Assay for Fluorescence polarization (FP) of Fl-ATP monitors HYPE-mediated AMPylation. FIG. 1A depicts FP AMPylation assay design, WT and E234G HYPE autoAMPylation reactions were measured for FP on separate 384-well microplates. Z′=0.67 and S/B=3.6. When unattached to WT HYPE or compound-inhibited E234G HYPE (purple), Fl-ATP undergoes rapid rotation to depolarize plane-polarized light, giving basal FP signal. Attachment of Fl-AMP via autoAMPylation (compound-activated WT HYPE or E234G HYPE) permits light to remain polarized for a high FP signal. FIG. 1B Structure of Fl-ATP (N6-(6-amino)hexyl-ATP-5-carboxyl-fluorescein). FIG. 1C Shows assay reproducibility assessment plot. Each dot represents a single AMPylation reaction in a separate well. Black dash lines represent three standard deviations (SD) plus or minus the control means.

FIG. 2 (FIG. 2A-C) depicts plots for WT HYPE activator HTS. FIG. 2A WT HYPE FP autoAMPylation reaction incubated with compounds from either DIVERset or CNS-Set libraries. All samples were normalized to WT and E234G HYPE controls on the same plate in the DMSO-containing buffer. Each dot represents a different compound incubated with a single AMPylation reaction in a separate well. Black dashes represent +/−3 SD from the mean. Red dashes represent the 20% hit definition, with all dots above this threshold being considered hit compounds. Minor tick marks on the x-axis represent a single 384 well plate. FIG. 2B Plate-to-plate variability of the high (E234G HYPE) and low (WT HYPE) DMSO internal controls. Left and right y-axes are for Z′ and S/B values, respectively. Each dot represents the calculated Z′ or S/B values from 32 E234G HYPE high controls and 32 WT HYPE low controls within a single plate. FIG. 2C Coefficient of variance assessment. Left and right y-axes are for WT and E234G HYPE CV (coefficient of variation), respectively. Each dot represents the calculated CV value from 32 E234G HYPE high controls and 32 WT HYPE low controls within a single plate.

FIG. 3 (FIG. 3A-C) shows WT HYPE activator validation. FIG. 3A Comparison between initial HTS compound activity (x-axis) and second pass, cherrypick (CP) validation (y-axis) with the same FP AMPylation assay. All validation compounds were assayed on the same plate and normalized to internal high (E234G HYPE) and low (WT HYPE) DMSO controls. Each dot represents the same compound incubated in two independent AMPylation reactions. FIG. 3B Data re-processed from FIG. 3A to show compound validation correlation differences in the DIVERset library. FIG. 3C Data re-processed from FIG. 3A to show compound validation correlation differences in the CNS-Set library.

FIG. 4 (FIG. 4A-D) shows Chemical structures of selected WT HYPE activators. FIG. 4A) Compound A2.5. FIG. 4B) Compound A2.6. FIG. 4C) Compound A2.7. FIG. 4D) Compound A2.8.

FIG. 5 (FIG. 5A-E) shows WT HYPE activator concentration-response curve. FIG. 5A FP plots from WT HYPE AMPylation reactions incubated with increasing concentrations of DMSO-dissolved activators from 0 to 1000 μM. All data were fitted to Equation 5 (see Methods) to determine EC50 values. (FIG. 5B-FIG. 5E) Reprocessed graphs of each individual activator (A2.5-A2.8) fitted to Equation 5 FIG. 5B A2.5, FIG. 5C A2.6, FIG. 5D A2.7, FIG. 5D A2.8.

FIG. 6 (FIG. 6A-D) shows WT HYPE activator validation and IbpA-2 cross reactivity using in-gel AMPylation assays. FIG. 6A Representative fluorescence in-gel autoAMPylation assay with WT HYPE or E234G HYPE (last lane). Fluorescence image (upper) shows direct protein AMPylation. Coomassie (lower) displays protein loading. FIG. 6B Quantification of data shown in 6A normalized to internal DMSO controls. Quantified data represented as the mean+/−SEM of four independent experiments. Unpaired t-tests were performed to determine statistical significance. FIG. 6C Representative radioactive in-gel AMPylation assay with WT IbpA-Fic2, Cdc42 and α-32P-ATP. WT HYPE-mediated AMPylation of T229A BiP-AMP with or without A1 was used as an activation control. Phosphor screen image (upper) shows direct protein AMPylation. Coomassie (lower) displays protein loading. FIG. 6D Quantification of data shown in 6C normalized to internal DMSO controls (first lane). Quantified data represented as the mean+/−SEM of three independent experiments. Unpaired t-tests were performed between DMSO (first lane) and all other IbpA-2+Cdc42 samples incubated with compound to determine statistical significance.

FIG. 7 (FIG. 7A-C) shows E234G HYPE inhibitor HTS. FIG. 7A E234G HYPE FP autoAMPylation reaction incubated with compounds from either DIVERset or CNS-Set libraries. All samples were normalized to WT and E234G HYPE controls on the same plate in DMSO-containing buffer. Each dot represents a different compound incubated with a single AMPylation reaction in a separate well. Black dashes represent +/−3 SD from the mean. Red dashes represent the 20% hit definition, with all dots above this threshold being considered hit compounds. Minor tick marks on the x-axis represent a single 384 well plate. FIG. 7B Plate-to-plate variability of the high (E234G HYPE) and low (WT HYPE) DMSO internal controls. Left and right y-axes are for Z′ and S/B values, respectively. Each dot represents the calculated Z′ or S/B values from 32 E234G HYPE high controls and 32 WT HYPE low controls within a single plate. FIG. 7C Coefficient of variance assessment. Left and right y-axes are for WT and E234G HYPE CV, respectively. Each dot represents the calculated CV value from 32 E234G HYPE high controls and 32 WT HYPE low controls within a single plate.

FIG. 8 (FIG. 8A-C) shows E234G HYPE inhibitor validation. FIG. 8A Comparison between initial HTS compound activity (x-axis) and second pass, cherrypick (CP) validation (y-axis) with the same FP AMPylation assay. All validation compounds were assayed on the same plate and normalized to internal high (E234G HYPE) and low (WT HYPE) DMSO controls. Each dot represents the same compound incubated in two independent AMPylation reactions. FIG. 8B Data re-processed from 8A to show compound validation correlation differences between in DIVERset library. FIG. 8C Data re-processed from 8A to show compound validation correlation differences between in CNS-Set library.

FIG. 9 (FIG. 9A-L) shows Chemical structures of selected E234G HYPE inhibitors. FIG. 9A) Compound I2.1. FIG. 9B) Compound I2.2. FIG. 9C) Compound I2.3. FIG. 9D) Compound I2.4. FIG. 9E) Compound I2.5. FIG. 9F) Compound I2.6. FIG. 9G) Compound I2.7. FIG. 9H) Compound I2.8. FIG. 9I) Compound I2.9. FIG. 9J) Compound I2.10. FIG. 9K) Compound I2.11. FIG. 9L) Compound I2.12.

FIG. 10 (FIG. 10A-C) shows E234G HYPE inhibitor concentration-response curve. FIG. 10A FP plots from E234G HYPE AMPylation reactions incubated with increasing concentrations of DMSO-dissolved inhibitors from 0 to 200 μM. All data were fitted to Equation 6 (see Methods) to determine IC50 values. FIG. 10B Reprocessed graphs of the top inhibitor, I2.10. FIG. 10C Reprocessed graph of the top inhibitor from the pilot HTS, I2, for comparison. All data were fitted to Equation 5 (see Methods) to determine IC50 values.

FIG. 11 (FIG. 11A-D) shows E234G HYPE inhibitor validation using in-gel AMPylation assays. FIG. 11A Representative fluorescence in-gel autoAMPylation assay with E234G HYPE or WT HYPE (last lane). Fluorescence image (upper) shows direct protein AMPylation. Coomassie (lower) displays protein loading. FIG. 11B Quantification of data from 11A normalized to internal DMSO controls. Quantified data represented as the mean+/−SEM of four independent experiments. Unpaired t-tests were performed: **=p<0.01. FIG. 11C Representative radioactive in-gel autoAMPylation assay with E234G HYPE or WT HYPE (last lane). Phosphor screen image (upper) shows direct protein AMPylation. Coomassie (lower) displays protein loading. FIG. 11D Quantification of data from 11C) normalized to internal DMSO controls. Quantified data represented as the mean+/−SEM of four independent experiments. Unpaired t-tests were performed: *=p<0.05;***=p<0.005.

FIG. 12 (FIG. 12A-D) Shows E234G HYPE inhibitor validation of BiP AMPylation with in-gel assays. FIG. 12A Representative fluorescence in-gel T229A BiP AMPylation assay with E234G HYPE or WT HYPE (last lane). Fluorescence image (upper) shows direct protein AMPylation. Coomassie (lower) displays protein loading. FIG. 12B Quantification of data from 12A normalized to internal DMSO controls. Quantified data represented as the mean+/−SEM of four independent experiments. Unpaired t-tests were performed: *=p<0.05. FIG. 12C Representative radioactive in-gel T229A BiP AMPylation assay with E234G HYPE or WT HYPE (last lane). Phosphor screen image (upper) shows direct protein AMPylation. Coomassie (lower) displays protein loading. FIG. 12D Quantification of data from FIG. 12C normalized to internal DMSO controls. Quantified data represented as the mean+/−SEM of four independent experiments. Unpaired t-tests were performed: *=p<0.05.

FIG. 13 (FIG. 13A-B) Cross reactivity of inhibitors on IbpA-2 AMPylation of Cdc42. FIG. 13A Representative radioactive in-gel AMPylation assay with WT IbpA-Fic2, Cdc42 and α-32P-ATP. E234G HYPE-mediated AMPylation of T229A BiP-AMP with or without I2 was used as an inhibition control. Phosphor screen image (upper) shows direct protein AMPylation. Coomassie (lower) displays protein loading. FIG. 13B Quantification of data from 13A normalized to internal DMSO controls (first lane). Quantified data represented as the mean+/−SEM of three independent experiments. Unpaired t-tests were performed between DMSO (first lane) and all other IbpA-2+Cdc42 samples incubated with compound to determine statistical significance.

FIG. 14 (FIG. 14A-B) shows Compound impact on HYPE-mediated deAMPylation. FIG. 14A Representative fluorescence in-gel deAMPylation assay with WT HYPE used a deAMPylase and T229A BiP-AMP used as a deAMPylation substrate. All T229A BiP was initially AMPylated by E234G HYPE and Fl-ATP to completion. Buffer/DMSO (first lane) and catalytically dead E234G/H363A (EG/HA) HYPE (last lane) served as negative controls for deAMPylation. (last lane). Fluorescence image (upper) shows direct protein de/AMPylation. Coomassie (lower) displays protein loading. FIG. 14B Quantification of data from 14A normalized to internal Buffer/DMSO controls (first lane). Quantified data represented as the mean+/−SEM of four independent experiments. Unpaired t-tests were performed between WT HYPE/DMSO (second lane) and all other samples incubated with compound to determine statistical significance.

FIG. 15 (FIG. 15A-B) shows Assessment of cellular toxicity FIG. 15A MTT cell viability assay of HeLa cells incubated with increasing concentrations of compounds for 48 hr on a microplate then treated with MTT reagents to produce colorimetric signal. Cell viability was calculated from colorimetric absorbance signal at 570 nm in accordance with Eq. 8 (see Methods). A1 was added as a positive control for toxicity. All experimental performed in three biological replicates with three technical replicates each. Quantified data represented as the mean+/−SEM of three independent replicates. FIG. 15B As in 15A but with HEK 293 cells.

FIG. 16 (FIG. 16A-D) Depicts Computer Modeling of the docked pose of I2.10 in the apo-HYPE structure was generated using Glide. Key residues in the binding site are shown as sticks, with polar hydrogens shown for clarity. Predicted protein-ligand hydrogen bonds are depicted as yellow dashes. (FIG. 16A) Docked pose for I2.10 (orange sticks) in apo-HYPE (gray cartoon). (FIG. 16B) Two-dimensional protein-ligand diagram for the docked pose of I2.10 in apo-HYPE with arrows to represent hydrogen bond directionality and green residues representing a hydrophobic microenvironment. (FIG. 16C) Docked pose for I2.10 (orange sticks) in apo-HYPE (gray surface). (FIG. 16D) Docked pose for I2.10 (orange sticks) in HYPE (gray surface) aligned with holo-ATP-bound (cyan sticks) HYPE (PDB: 4U07, pale green cartoon). Panels A, B, and D were generated using PyMol. The ligand-interaction diagram in panel B was generated in Maestro.

### DETAILED DESCRIPTION

While the concepts of the present disclosure are illustrated and described in detail in the figures and the description herein, results in the figures and their description are to be considered as exemplary and not restrictive in character; it being understood that only the illustrative embodiments are shown and described and that all changes and modifications that come within the spirit of the disclosure are desired to be protected.

As used herein, the following terms and phrases shall have the meanings set forth below. Unless defined otherwise, all technical and scientific terms used herein have the same meaning as commonly understood to one of ordinary skill in the art.

In the present disclosure the term “about” can allow for a degree of variability in a value or range, for example, within 10%, within 5%, or within 1% of a stated value or of a stated limit of a range. In the present disclosure, the term “substantially” can allow for a degree of variability in a value or range, for example, within 90%, within 95%, 99%, 99.5%, 99.9%, 99.99%, or at least about 99.999% or more of a stated value or of a stated limit of a range.

The term “substituted” as used herein refers to a functional group in which one or more hydrogen atoms contained therein are replaced by one or more non-hydrogen atoms. The term “functional group” or “substituent” as used herein refers to a group that can be or is substituted onto a molecule. Examples of substituents or functional groups include, but are not limited to, a halogen (e.g., F, Cl, Br, and I); an oxygen atom in groups such as hydroxyl groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, azides, hydroxylamines, cyano, nitro groups, N-oxides, hydrazides, and enamines; and other heteroatoms in various other groups.

The term “alkyl” as used herein refers to substituted or unsubstituted straight chain and branched alkyl groups and cycloalkyl groups having from 1 to about 20 carbon atoms (C1-C20), 1 to 12 carbons (C1-C12), 1 to 8 carbon atoms (C1-C8), or, in some embodiments, from 1 to 6 carbon atoms (C1-C6). Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to substituted or unsubstituted straight chain and branched divalent alkenyl and cycloalkenyl groups having from 2 to 20 carbon atoms (C2-C20), 2 to 12 carbons (C2-C12), 2 to 8 carbon atoms (C2-C8) or, in some embodiments, from 2 to 4 carbon atoms (C2-C4) and at least one carbon-carbon double bond. Examples of straight chain alkenyl groups include those with from 2 to 8 carbon atoms such as —CH═CH—, —CH═CHCH2-, and the like. Examples of branched alkenyl groups include, but are not limited to, —CH═C(CH3)- and the like.

An alkynyl group is the fragment, containing an open point of attachment on a carbon atom that would form if a hydrogen atom bonded to a triply bonded carbon is removed from the molecule of an alkyne. The term “hydroxyalkyl” as used herein refers to alkyl groups as defined herein substituted with at least one hydroxyl (—OH) group.

The term “cycloalkyl” as used herein refers to substituted or unsubstituted cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. In some embodiments, cycloalkyl groups can have 3 to 6 carbon atoms (C3-C6). Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like.

The term “acyl” as used herein refers to a group containing a carbonyl moiety wherein the group is bonded via the carbonyl carbon atom. The carbonyl carbon atom is also bonded to another carbon atom, which can be part of a substituted or unsubstituted alkyl, aryl, aralkyl cycloalkyl, cycloalkylalkyl, heterocyclyl, heterocyclylalkyl, heteroaryl, heteroarylalkyl group or the like. In the special case wherein the carbonyl carbon atom is bonded to a hydrogen, the group is a “formyl” group, an acyl group as the term is defined herein. An acyl group can include 0 to about 12-40, 6-10, 1-5 or 2-5 additional carbon atoms bonded to the carbonyl group. An acryloyl group is an example of an acyl group. An acyl group can also include heteroatoms within the meaning here. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “aryl” as used herein refers to substituted or unsubstituted cyclic aromatic hydrocarbons that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons (C6-C14) or from 6 to 10 carbon atoms (C6-C10) in the ring portions of the groups. Aryl groups can be unsubstituted or substituted, as defined herein. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed herein.

The term “aralkyl” and “arylalkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “heterocyclyl” as used herein refers to substituted or unsubstituted aromatic and non-aromatic ring compounds containing 3 or more ring members, of which, one or more is a heteroatom such as, but not limited to, B, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. In some embodiments, heterocyclyl groups include heterocyclyl groups that include 3 to 8 carbon atoms (C3-C8), 3 to 6 carbon atoms (C3-C6) or 6 to 8 carbon atoms (C6-C8).

A heteroaryl ring is an embodiment of a heterocyclyl group. The phrase “heterocyclyl group” includes fused ring species including those that include fused aromatic and non-aromatic groups. Representative heterocyclyl groups include, but are not limited to pyrrolidinyl, azetidinyl, piperidynyl, piperazinyl, morpholinyl, chromanyl, indolinonyl, isoindolinonyl, furanyl, pyrrolidinyl, pyridinyl, pyrazinyl, pyrimidinyl, triazinyl, thiophenyl, tetrahydrofuranyl, pyrrolyl, oxazolyl, oxadiazolyl, imidazolyl, triazyolyl, tetrazolyl, benzoxazolinyl, benzthiazolinyl, and benzimidazolinyl groups.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclylalkyl groups include, but are not limited to, furan-2-yl methyl, furan-3-yl methyl, pyridine-3-yl methyl, tetrahydrofuran-2-yl methyl, and indol-2-yl propyl.

The term “heteroarylalkyl” as used herein refers to alkyl groups as defined herein in which a hydrogen or carbon bond of an alkyl group is replaced with a bond to a heteroaryl group as defined herein.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can further include double or triple bonds, and can also include heteroatoms. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

The term “amine” as used herein refers to primary, secondary, and tertiary amines having, e.g., the formula N(group)3 wherein each group can independently be H or non-H, such as alkyl, aryl, and the like. Amines include but are not limited to R NH2, for example, alkylamines, arylamines, alkylarylamines; R2NH wherein each R is independently selected, such as dialkylamines, diarylamines, aralkylamines, heterocyclylamines and the like; and R3N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH2, —NHR, —NR2, —NR3+, wherein each R is independently selected, and protonated forms of each, except for —NR3+, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, —CF(CH3)2 and the like.

The term “optionally substituted,” or “optional substituents,” as used herein, means that the groups in question are either unsubstituted or substituted with one or more of the substituents specified. When the groups in question are substituted with more than one substituent, the substituents may be the same or different. When using the terms “independently,” “independently are,” and “independently selected from” mean that the groups in question may be the same or different. Certain of the herein defined terms may occur more than once in the structure, and upon such occurrence each term shall be defined independently of the other.

The compounds described herein may contain one or more chiral centers, or may otherwise be capable of existing as multiple stereoisomers. It is to be understood that in one embodiment, the disclosure described herein is not limited to any particular stereochemical requirement, and that the compounds, and compositions, methods, uses, and medicaments that include them may be optically pure, or may be any of a variety of stereoisomeric mixtures, including racemic and other mixtures of enantiomers, other mixtures of diastereomers, and the like. It is also to be understood that such mixtures of stereoisomers may include a single stereochemical configuration at one or more chiral centers, while including mixtures of stereochemical configuration at one or more other chiral centers.

Similarly, the compounds described herein may include geometric centers, such as cis, trans, E, and Z double bonds. It is to be understood that in another embodiment, the disclosure described herein is not limited to any particular geometric isomer requirement, and that the compounds, and compositions, methods, uses, and medicaments that include them may be pure, or may be any of a variety of geometric isomer mixtures. It is also to be understood that such mixtures of geometric isomers may include a single configuration at one or more double bonds, while including mixtures of geometry at one or more other double bonds.

As used herein, the term “salts” and “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound which contains a basic or acidic moiety by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, the disclosure of which is hereby incorporated by reference.

The term “solvate” means a compound, or a salt thereof, that further includes a stoichiometric or non-stoichiometric amount of solvent bound by non-covalent intermolecular forces. Where the solvent is water, the solvate is a hydrate.

Further, in each of the foregoing and following embodiments, it is to be understood that the formulae include and represent not only all pharmaceutically acceptable salts of the compounds, but also include any and all hydrates and/or solvates of the compound formulae or salts thereof. It is to be appreciated that certain functional groups, such as the hydroxy, amino, and like groups form complexes and/or coordination compounds with water and/or various solvents, in the various physical forms of the compounds. Accordingly, the above formulae are to be understood to include and represent those various hydrates and/or solvates. In each of the foregoing and following embodiments, it is also to be understood that the formulae include and represent each possible isomer, such as stereoisomers and geometric isomers, both individually and in any and all possible mixtures. In each of the foregoing and following embodiments, it is also to be understood that the formulae include and represent any and all crystalline forms, partially crystalline forms, and non-crystalline and/or amorphous forms of the compounds.

As used herein, a “pharmaceutical formulation” is a product which contains an active ingredient which may be a particular compound of a chemical formula. A pharmaceutical formulation may contain one or more active ingredient along with one or more suitable carrier, excipient or diluent. A pharmaceutical formulation may have one or more therapeutic effect on a subject to be treated there with. A pharmaceutical formulation may be adapted for different storage, transport of administration methods.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As used herein, the term “administering” includes all means of introducing the compounds and compositions described herein to the patient, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically acceptable carriers, adjuvants, and vehicles.

Illustrative formats for oral administration include tablets, capsules, elixirs, syrups, and the like. Illustrative routes for parenteral administration include intravenous, intraarterial, intraperitoneal, epidural, intraurethral, intrasternal, intramuscular and subcutaneous, as well as any other art recognized route of parenteral administration.

Illustrative means of parenteral administration include needle (including microneedle) injectors, needle-free injectors and infusion techniques, as well as any other means of parenteral administration recognized in the art. Parenteral formulations are typically aqueous solutions which may contain excipients such as salts, carbohydrates and buffering agents (preferably at a pH in the range from about 3 to about 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilization, may readily be accomplished using standard pharmaceutical techniques well known to those skilled in the art. Parenteral administration of a compound is illustratively performed in the form of saline solutions or with the compound incorporated into liposomes. In cases where the compound in itself is not sufficiently soluble to be dissolved, a solubilizer such as ethanol can be applied.

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage used.

It is to be understood that in the methods described herein, the individual components of a co-administration, or combination can be administered by any suitable means, contemporaneously, simultaneously, sequentially, separately or in a single pharmaceutical formulation. Where the co-administered compounds or compositions are administered in separate dosage forms, the number of dosages administered per day for each compound may be the same or different. The compounds or compositions may be administered via the same or different routes of administration. The compounds or compositions may be administered according to simultaneous or alternating regimens, at the same or different times during the course of the therapy, concurrently in divided or single forms.

The term “therapeutically effective amount” as used herein, refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill.

Depending upon the route of administration, a wide range of permissible dosages are contemplated herein, including doses falling in the range from about 1 μg/kg to about 1 g/kg. The dosages may be single or divided, and may administered according to a wide variety of protocols, including q.d. (once a day), b.i.d. (twice a day), t.i.d. (three times a day), or even every other day, once a week, once a month, once a quarter, and the like. In each of these cases it is understood that the therapeutically effective amounts described herein correspond to the instance of administration, or alternatively to the total daily, weekly, month, or quarterly dose, as determined by the dosing protocol.

In addition to the illustrative dosages and dosing protocols described herein, it is to be understood that an effective amount of any one or a mixture of the compounds described herein can be determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

The term “patient” includes human and non-human animals such as companion animals (dogs and cats and the like) and livestock animals. Livestock animals are animals raised for food production. The patient to be treated is preferably a mammal, in particular a human being.

The present disclosure provides for methods for treating neurodegenerative diseases and/or disorders. In a further embodiment the present disclosure provides for methods for treating neurodegenerative diseases and/or disorders which are impacted by HYPE-mediated AMPylation. Such neurodegenerative diseases include and are not limited to Alzheimer's Disease, Huntington's disease, Parkinson's disease. As defined by the NIH NCI neurodegenerative disorder is a type of disease in which cells of the central nervous system stop working or die. Neurodegenerative disorders usually get worse over time and have no cure. They may be genetic or be caused by a tumor or stroke. Neurodegenerative disorders also occur in people who drink large amounts of alcohol or are exposed to certain viruses or toxins. Examples of neurodegenerative disorders include Alzheimer's disease and Parkinson's disease.

The present application relates generally to a method to treat a patient with compounds which moderate HYPE-mediated AMPylation and/or pharmaceutically acceptable salts thereof. In a preferred embodiment the compounds are specific for HYPE-mediated activity when compared with other cellular processes.

The present disclosure relates to a method for treating a patient of neurodegenerative disorder impacted by HYPE-mediated AMPylation. In some illustrative embodiments, the present disclosure relates to a method for treating a patient of Alzheimer's Disease, Parkinson's Disease, Huntington's Disease, or neurodegenerative disorder impacted by HYPE-mediated AMPylation. In some illustrative embodiments, the present disclosure relates to a method for treating a patient of Alzheimer's Disease. In some illustrative embodiments, the present disclosure relates to a method for treating a patient of Parkinson's Disease. In some illustrative embodiments, the present disclosure relates to a method for treating a patient of Huntington's Disease. In some illustrative embodiments, the present disclosure relates to a method for treating a patient of a neurodegenerative disorder impacted by HYPE-mediated AMPylation. The present disclosure relates to a method for treating a patient of a neurodegenerative. The present disclosure relates to a method for treating a patient impacted by HYPE-mediated AMPylation.

The present disclosure also provides for Pharmaceutical Compositions for treating neurodegenerative diseases and/or disorders which are impacted by HYPE-mediated AMPylation.

The present disclosure provides for compounds which are useful for moderating, altering, inhibiting, and/or interfering with HYPE-mediated AMPylation. This moderation encompasses the disruption of the activity of HYPE-mediated AMPylation. Such disruption may be accomplished by abolition of the activity via competitive inhibition, steric inhibition or the like. Also provided for are compounds which are useful for moderating, altering, and/or interfering with HYPE-mediated AMPylation where such moderation occurs via binding, steric conformational changes or the like which stimulate or over-stimulate HYPE-mediated AMPylation resulting in desired physiological effect.

The present disclosure provides for methods of screening for such compounds.

A method for screening for an activator of HYPE-mediated AMPylation comprising; using hypoactive AMPylase WT HYPE and Mg Buffer to screen for potential activators.

A method for screening for inhibitors of HYPE-mediated AMPylation comprising; using hyperactive E234G HYPE and Mg Buffer to screen for potential inhibitors.

The present disclosure provides for a compound having a formula (I)

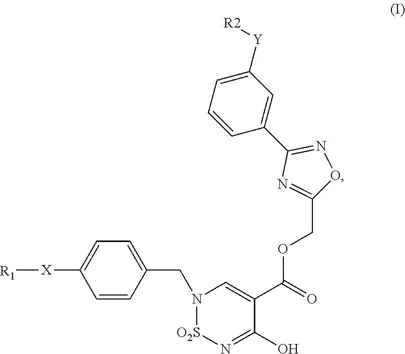

or a pharmaceutically acceptable salt thereof, wherein
X is H or O;
Y is H or O;
R1 is a C1-C12 alkyl;
R2 is a C1-C12 alkyl; heteroalkyl, heteroalkenyl, heteroalkynyl, cycloalkyl, heterocyloalkyl, cycloalkenyl, heterocycloakenyl, heterocyclyl, or cycloalkene.

The present disclosure provides for compounds of the formula

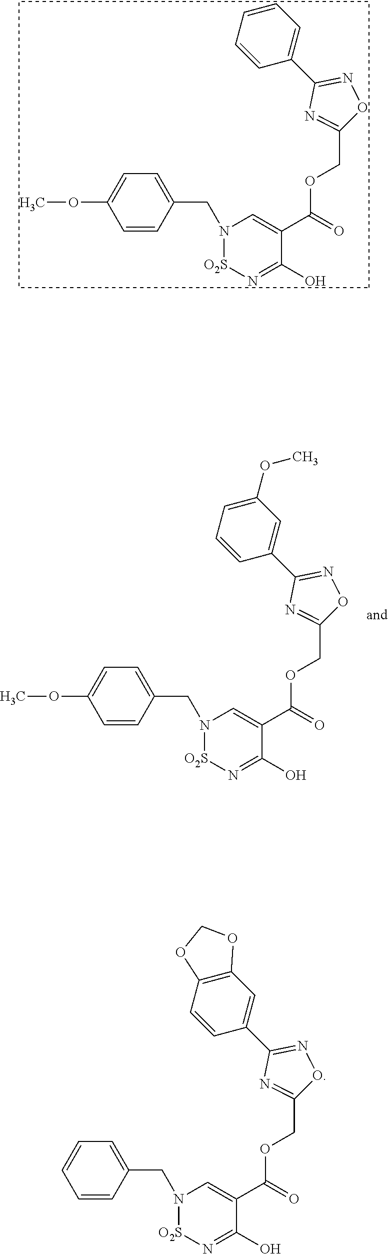 [SMILES: COc1ccc(CN2C=C(C(=O)OCc3nc(-c4ccccc4)no3)C(O)=NS2(=O)=O)cc1 ; COc1ccc(CN2C=C(C(=O)OCc3nc(-c4cccc(OC)c4)no3)C(O)=NS2(=O)=O)cc1 ; O=C(OCc1nc(-c2ccc3c(c2)OCO3)no1)C1=CN(Cc2ccccc2)S(=O)(=O)N=C1O]

The present disclosure also provides from compounds of the formula

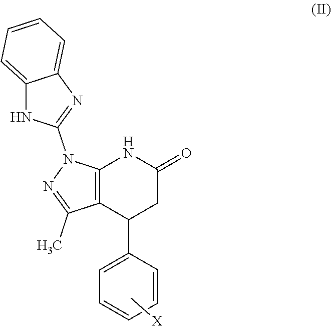

or a pharmaceutically acceptable salt thereof, wherein
X is 1 or 2 optionally bound halo-substituent to the ring structure.

In particular the present disclosure provides for compounds of the formula

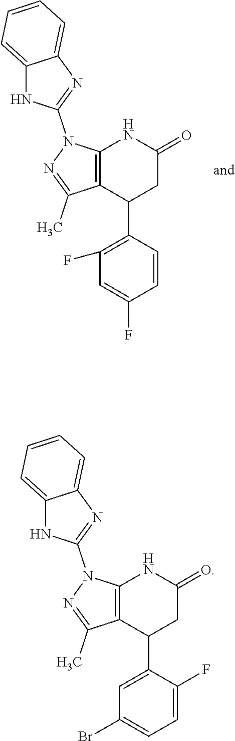 [SMILES: Cc1nn(-c2nc3ccccc3[nH]2)c2c1C(c1ccc(F)cc1F)CC(=O)N2 ; Cc1nn(-c2nc3ccccc3[nH]2)c2c1C(c1cc(Br)ccc1F)CC(=O)N2]

The present disclosure provides for a compound of formula III

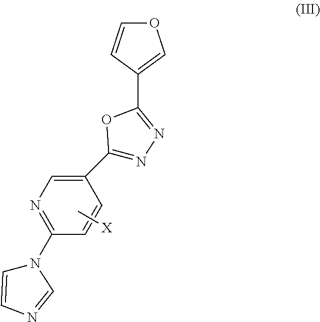

or a pharmaceutically acceptable salt thereof, wherein
X is 1 or 2 optionally bound halo-substituent to the ring structure.

The present disclosure provides for compounds of the formula

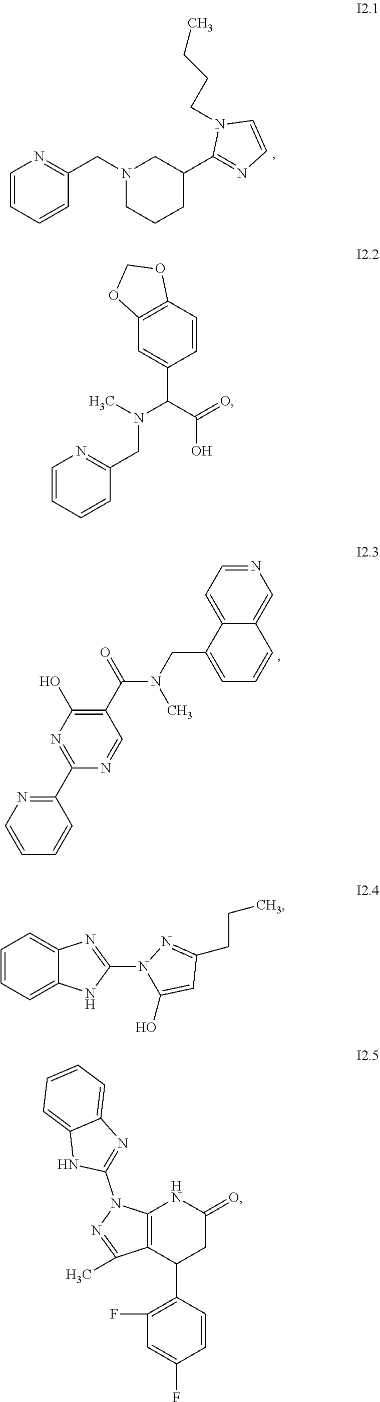

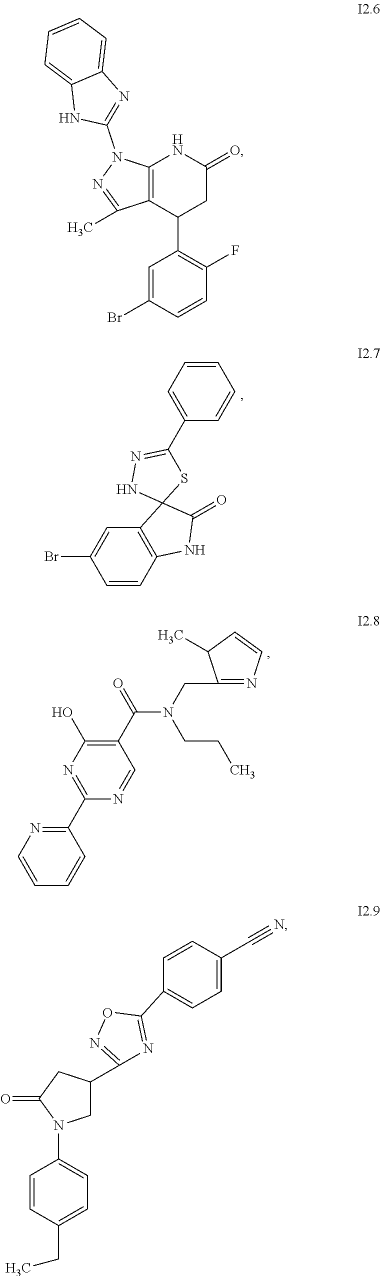

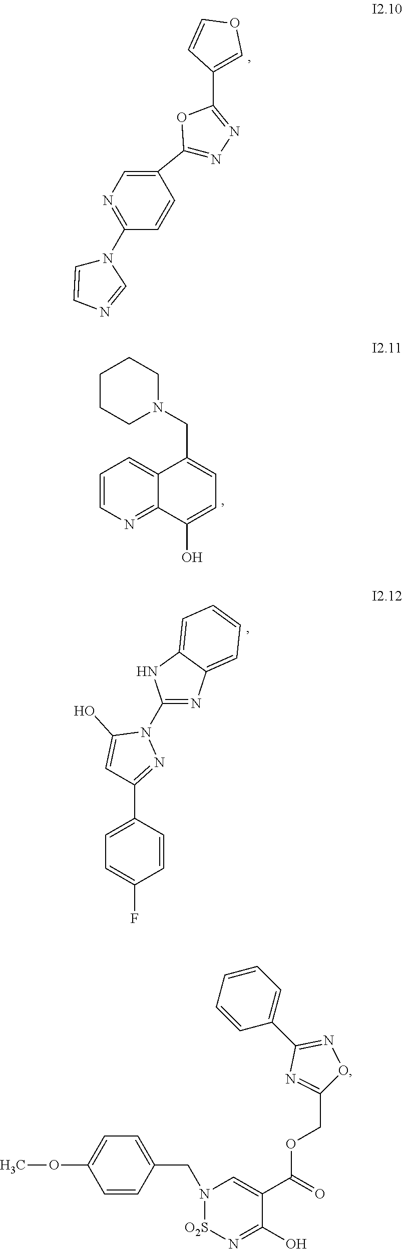 [SMILES: c1cn(-c2ccc(-c3nnc(-c4ccoc4)o3)cn2)cn1 ; Oc1ccc(CN2CCCCC2)c2cccnc12 ; Oc1cc(-c2ccc(F)cc2)nn1-c1nc2ccccc2[nH]1 ; COc1ccc(CN2C=C(C(=O)OCc3nc(-c4ccccc4)no3)C(O)=NS2(=O)=O)cc1]

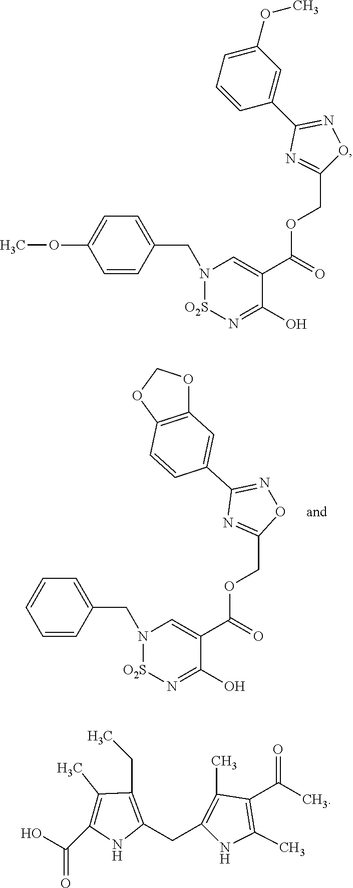 [SMILES: COc1ccc(CN2C=C(C(=O)OCc3nc(-c4cccc(OC)c4)no3)C(O)=NS2(=O)=O)cc1 ; O=C(OCc1nc(-c2ccc3c(c2)OCO3)no1)C1=CN(Cc2ccccc2)S(=O)(=O)N=C1O ; CCc1c(Cc2[nH]c(C)c(C(C)=O)c2C)[nH]c(C(=O)O)c1C]

In some other illustrative embodiments, the present disclosure relates to a pharmaceutical composition for the treatment of neurodegenerative disease comprising administering therapeutically effective amount of a compound selected from those described above.

In a further embodiment, the described compound may be combined with one or more anti-inflammatory agents or other therapeutic agent, with one or more pharmaceutically acceptable carriers, diluents, and excipients.

In some other illustrative embodiments, the present disclosure relates to a pharmaceutical composition for the treatment neurodegenerative disorder. In further embodiments the present disclosure relates to the treatment of Alzheimer's Disease. In further embodiments the present disclosure relates to the treatment of Huntington's Disease.

In some other illustrative embodiments, the present disclosure relates to a pharmaceutical composition for the treatment of neurodegenerative disease are administered consequentially.

The term “patient” includes human and non-human animals such as companion animals (dogs and cats and the like) and livestock animals. Livestock animals are animals raised for food production. The patient to be treated is preferably a mammal, in particular a human being.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffered solutions; and (21) other non-toxic compatible substances employed in pharmaceutical formulations.

As used herein, the term “administering” includes all means of introducing the compounds and compositions described herein to the patient, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically acceptable carriers, adjuvants, and vehicles.

It is to be understood that the total daily usage of the compounds and compositions described herein may be decided by the attending physician within the scope of sound medical judgment. The specific therapeutically effective dose level for any particular patient will depend upon a variety of factors, including the disorder being treated and the severity of the disorder; activity of the specific compound employed; the specific composition employed; the age, body weight, general health, gender, and diet of the patient: the time of administration, and rate of excretion of the specific compound employed, the duration of the treatment, the drugs used in combination or coincidentally with the specific compound employed; and like factors well known to the researcher, veterinarian, medical doctor or other clinician of ordinary skill.

Depending upon the route of administration, a wide range of permissible dosages are contemplated herein, including doses falling in the range from about 1 μg/kg to about 1 g/kg. The dosage may be single or divided, and may be administered according to a wide variety of dosing protocols, including q.d., b.i.d., t.i.d., or even every other day, once a week, once a month, and the like. In each case the therapeutically effective amount described herein corresponds to the instance of administration, or alternatively to the total daily, weekly, or monthly dose.

As used herein, the term “therapeutically effective amount” refers to that amount of active compound or pharmaceutical agent that elicits the biological or medicinal response in a tissue system, animal or human that is being sought by a researcher, veterinarian, medical doctor or other clinicians, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment.

As used herein, the term “therapeutically effective amount” refers to the amount to be administered to a patient, and may be based on body surface area, patient weight, and/or patient condition. In addition, it is appreciated that there is an interrelationship of dosages determined for humans and those dosages determined for animals, including test animals (illustratively based on milligrams per meter squared of body surface) as described by Freireich, E. J., et al., Cancer Chemother. Rep. 1966, 50 (4), 219, the disclosure of which is incorporated herein by reference. Body surface area may be approximately determined from patient height and weight (see, e.g., Scientific Tables, Geigy Pharmaceuticals, Ardley, New York, pages 537-538 (1970)). A therapeutically effective amount of the compounds described herein may be defined as any amount useful for inhibiting the growth of (or killing) a population of malignant cells or cancer cells, such as may be found in a patient in need of relief from such cancer or malignancy. Typically, such effective amounts range from about 5 mg/kg to about 500 mg/kg, from about 5 mg/kg to about 250 mg/kg, and/or from about 5 mg/kg to about 150 mg/kg of compound per patient body weight. It is appreciated that effective doses may also vary depending on the route of administration, optional excipient usage, and the possibility of co-usage of the compound with other conventional and non-conventional therapeutic treatments, including other anti-tumor agents, radiation therapy, and the like.

The present disclosure may be better understood in light of the following non-limiting compound examples and method examples.

AMPylation—the covalent transfer of an AMP from ATP onto a target protein—is catalyzed by the human enzyme HYPE to regulate its substrate, the ER heat shock chaperone BiP. HYPE-mediated AMPylation of BiP is critical for maintaining proteostasis in the ER and mounting a proper UPR (unfolded protein response) in times of proteostatic imbalance. Thus, manipulating HYPE's enzymatic activity is a key therapeutic strategy towards the treatment of various protein misfolding diseases. Herein, we present an optimized, fluorescence polarization-based, high-throughput screening (HTS) assay to discover activators and inhibitors of HYPE-mediated AMPylation. After challenging our HTS assay with over 30,000 compounds, we discovered a novel AMPylase inhibitor, I2.10. We also determined a low micromolar IC50 for I2.10 and employed biorthogonal counterscreens to validate its efficacy against HYPE's AMPylation of BiP. Further, we report low cytotoxicity of I2.10 on human cell lines. We thus established an optimized, high-quality HTS assay amenable to tracking HYPE's enzymatic activity at scale, and first novel compound capable of perturbing HYPE-directed AMPylation of BiP.

### Methods

### Protein Expression and Purification

Recombinant His6-SUMO tagged proteins were purified as previously described6. Specifically, Δ102 (aa 103-458) HYPE constructs (WT, E234G, and E234G/H363A) and Δ19 T229A BiP (aa 20-654) proteins were cloned into pSMT3 plasmids and expressed in E. coli BL21-DE3-RILP (Stratagene) in LB medium containing 50 μg/ml of kanamycin) to an optical density of A600=0.6. Protein expression was induced with 0.4 mM IPTG for 12-16 h at 18° C. Lysis was performed on frozen pellets dissolved in lysis buffer (50 mM Tris, 250 mM NaCl, 5 mM imidazole, 1 mM PMSF, pH 7.5). Lysed cells were centrifuged at 15,000 g for 50 min. Supernatants were poured over cobalt resin. Resin was washed with wash buffer (50 mM Tris, 250 mM NaCl, 15 mM imidazole, pH 7.5). Tagged protein were eluted with elution buffer (50 mM Tris, 250 mM NaCl, 350 mM imidazole, pH 7.5). The His6-SUMO tags were cleaved by incubating proteins with ULP1 overnight at 4° C. The protein mixture was diluted in wash buffer without imidazole and re-applied unto a cobalt column. The flow through containing cleaved protein was further purified by size exclusion chromatography in a buffer containing 100 mM Tris and 100 mM NaCl, pH 7.5. Fractions containing HYPE were verified for purity by SDS-PAGE and pooled together. Protein concentrations were measured spectrophotometrically at A280. Proteins were flash frozen and stored at −80° C. in Storage Buffer [50 mM Tris, 300 mM NaCl, 10% (v/v) glycerol, pH 7.5].

GST-tagged WT IbpA-Fic2 (IbpA-2) and Q61L Cdc42 were bacterially expressed and purified as previously described (Mattoo et al. 2011).

### Assay Development

### Microplate Optimization

Buffer optimization experiments were performed by incubating 0.4 μM of Δ102 HYPE enzyme with 25 nM Fl-ATP (final concentrations) in HTS buffer (50 mM HEPES (pH 7.5), 1 mM MgCl2, 0.1% Triton X-100, 1% DMSO) in the dark at room temperature for 10 min in 384-well black bottom, black wall microplates (20 μL reaction volume). Where specified, 1 mM MgCl2 was replaced with an equivalent concentration of MnCl2, or 0.1% Triton X-100 was replaced with 0.05% or 0.01% Trion X-100. Microplates were snap centrifuged at 1000 g for 10 sec. Microplates were loaded onto a BioTek BioStack NEO2 plate-reader and assessed for fluorescence polarization with 485/530 nm filters and a 55/50 gain adjustment.

A Multidrop 384 reagent dispenser (ThermoFisher) was used to add 0.4 μM HYPE, then 25 nM Fl-ATP (final concentrations) to 384-well black/black microplates (20 μL reaction volume). Both enzyme and nucleotide were dissolved in HTS Buffer. AMPylation Reactions were incubated for 10 min at room temperature in the dark. Microplates were snap centrifuged at 1000 g for 10 sec, then transferred to BioTek BioStack NEO2 plate-reader. Reactions were assessed for fluorescence polarization with 485/530 nm filters and a 55/50 gain adjustment. Z′, S/B, and CV values were determined by fitting the data to Eq. 1, 2, and 3, respectively,

$${Z}^{′} = 1 - \frac{3 ( {σ}_{p} + {σ}_{n} )}{\vert {μ}_{p} - {μ}_{n} \vert} \qquad ( 1 )$$

$$S / B = \frac{{μ}_{p}}{{μ}_{n}} \qquad ( 2 )$$

$$CV = \frac{σ}{μ} \times 100 \qquad ( 3 )$$

where μp and μn are the means of the positive (E234G HYPE) and negative (WT HYPE) controls, respectively; and σp and σn are the standard deviations of the positive and negative controls, respectively.

### High-Throughput Screen (HTS)

An Echo Liquid Handler (Labcyte) was used to transfer 200 nL of DMSO-dissolved compounds via acoustic-coupled ejection from 10 mM source plates into 384-well black, flat-bottom, black-walled microplates, for a final compound concentration of 10 μM. Compounds were sourced from the following libraries: DIVERset (Chembridge), CNS-Set (Chembridge). The compounds went into columns 3-22 of each plate, while columns 1, 2, 23, and 24 were reserved for equivalent volume of 100% DMSO controls.

A Multidrop 384 reagent dispenser was used to pipette 0.4 μM of WT HYPE (final concentration) dissolved in HTS Buffer into columns 1-22 of the activator plates, and columns 1 and 2 of the inhibitor plates as positive controls. 0.4 μM of E234G HYPE was similarly added to columns 3-24 of the inhibitor plates, and columns 23 and 24 of the inhibitor plates as positive controls. Enzymes were then incubated with either compounds or DMSO for 10 min at room temperature.

A Multidrop 384 reagent dispenser was used to pipette 25 nM of Fl-ATP (final concentration) into all wells of each plate, giving a final reaction volume of 20 μL. Plates were then incubated for 10 min at room temperature in the dark, followed by a snap centrifugation at 1000 g for 10 sec. Microplates were loaded onto BioTek BioStack stacker and read in succession for fluorescence polarization with 485/530 nm filters and a 55/50 gain adjustment. Fluorescence polarization values were converted to fractional activation (FA) or fractional inhibition FI) values according to Eq. (3) or (4), respectively,

$$FA = \frac{( x - {μ}_{n} )}{{μ}_{n}} \qquad ( 4 )$$

$$FI = \frac{( x - {μ}_{p} )}{{μ}_{p}} \qquad ( 5 )$$

where μp and μn are the means of the positive (E234G HYPE) and negative (WT HYPE) controls, respectively; and x is the measured value of fluorescence polarization.

Microplate “cherrypicking” validations were performed as described above.

### Concentration-Response Curves

A Multidrop 384 reagent dispenser was used to pipette 0.4 μM of WT or E234G HYPE (final concentrations) dissolved in HTS Buffer into designated microplate wells. 10 μM of DMSO-dissolved compounds (final concentrations) or equivalent volume of 100% DMSO were manually pipetted into their specified wells. Enzymes were incubated with compounds or DMSO for 10 min at room temperature. Reagent dispenser was then used to pipette 25 nM of Fl-ATP (final concentration) into each well, followed by a 10 min incubation at room temperature in the dark. Plates were snap centrifuged at 1000 g for 10 sec, then transferred to BioTek BioStack NEO2 plate-reader. Reactions were assessed for fluorescence polarization with 485/530 nm filters and a 55/50 gain adjustment. EC50 and IC50 values were determined by fitting polarization values to Eq. (5) and (6), respectively,

$$Y = \frac{( {Y}_{max} \times {[ I ]}^{n} )}{( {EC}_{50}^{n} + {[ I ]}^{n} )} + {Y}_{min} \qquad ( 6 )$$

$$Y = \frac{( {Y}_{max} \times {[ I ]}^{n} )}{( {IC}_{50}^{n} + {[ I ]}^{n} )} + {Y}_{min} \qquad ( 7 )$$

where Ymax is the maximum polarization signal; I is the μM concentration of activator or inhibitor; n is the Hill slope; EC50 or IC50 are the inflection point concentrations; and Ymin is the baseline polarization response.

### Fluorescence In-Gel AMPylation Validation

1 μM of WT HYPE (final concentration) was preincubated with 10 μM DMSO-dissolved compound (final concentration) or equivalent volume of 100% DMSO at room temperature for 10 min. Reactions were run for 20 hr at 30° C. in the dark and began with the addition of 1 μM Fl-ATP (final concentration). Alternatively, E234G HYPE AMPylation reactions were run at 10 min due to its hyperactivity. All other parameters were held constant. All reactions were performed HTS Buffer (50 mM HEPES, 1 mM MgCl2, 0.1% Triton X-100, pH 7.50). All reactions were quenched with 4×SDS loading dye, boiled for 5 min at 95° C., and samples were run on 12% SDS-PAGE gels. Gels were imaged on a Typhoon 9500 FLA imager (General Electric) for fluorescence at 473 nm. Imaged gels were then stained with Coomassie Brilliant Blue, destained, and imaged for protein concentration. Where specified, 5 μM of T229A BiP (final concentration) was added to reactions before the addition of compound.

### Radiolabeled In-Gel AMPylation Validation

1 μM of HYPE (final concentration) and (where specified) 5 μM of T229A BiP (final concentration) were preincubated with 10 μM DMSO-dissolved compound (final concentration) or equivalent volume of 100% DMSO at room temperature for 10 min in HTS Buffer. Reactions were run for 10 min at 30° C. and began with the addition of 0.01 μCi/μL α-32P-ATP (final concentration). All reactions were performed according to our previously described protocol for in vitro radioactive AMPylation assays19. Where specified, 5 μM of T229A BiP (final concentration) was added to reactions before the addition of compound.

All bacterial Fic specificity assays were conducted with 1 μM GST-tagged IbpA-Fic2, 5 μM GST-tagged Q61L Cdc42, and 0.01 μCi/μL α-32P-ATP (all final concentrations). All other reaction parameters were kept as per HYPE radiolabeled in-gel AMPylation assays.

### MTT Cell Viability Validation

The cell lines were procured from ATCC (American Type Culture Collection) and cultured in DMEM (Dulbecco's Modified Eagle Medium) supplemented with 10% FBS (fetal bovine serum) without antibiotics. Cells were grown and maintained in a humidified incubator at 37° C. with 5% CO2. For the MTT assay, cells were trypsinized and harvested at ˜80-90% confluency and seeded in 96 well plates at a density of 1×104 cells per well. The seeded cells were incubated overnight and treated with 1 μM, 3 μM, 5 μM, 7 μM, 10 μM, 20 μM, 30 μM or 50 μM (final concentration) of DMSO-dissolved compound for 48 hr. To rule out vehicle induced toxicity, only 0.5% of final DMSO concentration was added to the cells. Post-incubation, the compound treatment was removed and replaced with 0.5 mg/ml (final concentration) of freshly prepared MTT solution. After incubating for 3.5 hr at 37° C., 5% CO2 humidified incubator, MTT was carefully removed without aspirating the formazan crystals and replaced with equivalent volume of DMSO to dissolve the formazan crystals. The plates were then read using a multi-plate reader at 570 nm. The absorbance was recorded and % viability was calculated according to the following equation:

$$\% viability = \frac{{Ab}_{t}}{{Ab}_{c}} \times 100 \qquad ( 8 )$$

where, Abt is the absorbance of compound treated cells and Abc is the absorbance of control cells.

### Protein Preparation and Docking

All protein and ligand preparation and docking studies were run in the Schrodinger Small Molecule Drug Discovery Suite (Schrodinger, LLC, New York, NY, software release 2022-2) using computational tools available through its Maestro interface. The apo-HYPE (PDB: 4U04) crystal structure was prepared as follows. A protein reliability report was generated prior to preparation as a point of reference to ensure the improvement of the prepared structure. During the pre-processing step, termini were capped, missing side chains were filled in, bond orders were assigned, the CCD database was used, hydrogens were replaced, zero-order bonds to metals were created, disulfide bonds were created, missing loops were filled in using Prime (https://onlinelibrary.wiley.com/doi/10.1002/prot.10613; https://www.sciencedirect.com/science/article/pii/S0022283602004709?via%3Dihub), and heteroatom states were generated with pH 7.4±1.0 using Epik. The preprocessed structure was then optimized by sampling water orientations using PROPKA pH 7.4. Minimization of the optimized structure was then performed by converging heavy atoms to an RMSD of 0.3 Å using the OPLS4 forcefield, followed by removal of waters with less than two hydrogen bonds to non-waters. A protein reliability report was generated for the final prepared structure to ensure no structural problems existed in proximity to the binding pocket.

The hit compound I2.10 was prepared using LigPrep where possible heteroatom states were generated for pH 7.4±1.0 using Epik. The rest of the LigPrep parameters were left at default settings.

A receptor grid was generated by defining the enclosing box at the centroid of the selected residues H363, N369, Y400, and N407. The prepared I2.10 structure was then docked into the prepared apo-HYPE structure using the receptor grid with Glide, using Extra Precision (XP) docking and no constraints, with the remaining docking settings at default.

### Assay Design and Optimization

Though endogenously expressed HYPE exhibits dual catalytic activity, in vitro, WT HYPE is largely restricted to deAMPylation (Preissler et al 2017). These basal levels of AMPylase activity make WT HYPE an ideal candidate for discovering activators (Sanyal et al. 2015; Camara et al. 2020). Conversely, HYPE is rendered a hyperactive AMPylase upon mutation of the regulatory glutamate to glycine—E234G HYPE (Engel et al. 2012). Mechanistically, E234 regulates AMPylation by forming a salt bridge with R374, which in turn, precludes an AMPylation-competent orientation of ATP in the Fic domain active site. These catalytic differences were exploited in our HTS assay design by using hypoactive AMPylase WT HYPE to screen for potential activators and hyperactive E234G as the basis for discovering inhibitors. WT and E234G HYPE's relative AMPylation efficiencies remain similar for substrate and autoAMPylation. We, therefore, were able to conduct a streamlined, efficient screen with HYPE as its own substrate.

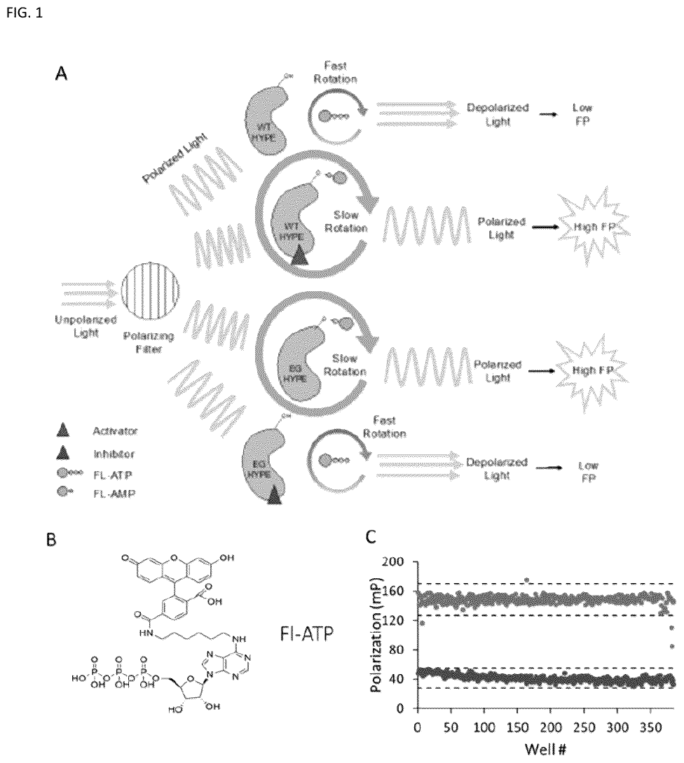

As a nucleotide source, we turned to a fluorescently polarizable (FP) ATP analog, Fl-ATP (FIG. 1A) (Camara et al. 2020; Lewallen et al. 2015; Lewallen et al. 2012). After light is polarized by a filter, the rapid rotation of free Fl-ATP in solution will depolarize this light, leading to low FP signal. However, if Fl-AMP is attached to a large protein (i.e., via AMPylation), this rotation is considerably slower and remains polarized with high FP signal. Of note, covalent attachment of Fl-AMP to a protein substrate is not the only way to slow down its rotation. Some compounds are prone to assembling in large aggregates which can trap Fl-ATP to produce a false positive AMPylation signal. To control for this potentially interfering occurrence, we added Triton X-100 (TX-100). This nonionic detergent is often used in small molecule screens to prevent aggregates from forming. However, an excess amount of detergent could have a deleterious effect on protein stability and catalysis. We, therefore, assayed a range of TX-100 concentrations to assess their impact on AMPylation, starting with 0.1%, equal to that of our pilot screen validation buffer (FIG. S1A). While 0.1% TX-100 proved suitable for our validation reactions, we wanted to ensure it would not interfere with AMPylation on our FP microplate screening format. We see that all measured concentrations yield acceptably high levels of E234G HYPE AMPylation and low levels of WT HYPE AMPylation. In fact, we find decreasing levels of detergent to enact a slight inhibitory effect on E234G HYPE AMPylation while WT signal is slightly boosted at the lowest concentration. These marginal catalytic differences prompted us to use a 0.1% TX-100 concentration, which would minimize the chance of compound aggregates.

HYPE-directed AMPylation is dependent on a divalent cation as a cofactor. Via an in-gel, in vitro AMPylation using α-32P-ATP we previously established a hierarchy of catalytic efficiencies among divalent cations with manganese (Mn2+) at the top, followed by magnesium (Mg2+)4. Despite the apparent prevalence of Mg2+, differences can arise between the assay formats (in-gel versus FP microplate) and nucleotide source (radiolabeled versus fluorescently labelled). Moreover, recent structures of HYPE and in complex with its substrate BiP elucidate an essential role for Mg2+ in coordinating the alpha and beta phosphates of ATP22,23. In direct assessment of cofactor preference, we conducted side-by-side WT and E234G HYPE FP microplate AMPylation reactions differing only by the presence of Mn2+ versus Mg2+. Interestingly, we observe a slight signal increase in the Mg2+-containing buffer over the one with Mn2+ for E234G HYPE AMPylation, and the opposite for WT HYPE (i.e., higher Mn2+ signal than Mg2+; FIG. S1B). During buffer optimization for our pilot screen, we noticed a similar trend for WT HYPE AMPylation, although there was no significant difference in E234G HYPE AMPylation between the cations (Camara et al. 2020). These differences could result from the presence of TX-100, which was absent in our pilot HTS screening buffer.

### Quality Assessment and Library Selection

After establishing optimal buffer conditions, we wanted to assess the quality and scalability of this assay for a full, automated HTS campaign. To this end, we turned to Z′ and signal/background (S/B) analysis (FIG. 1C) 24. We plated 384 WT and 384 E234G HYPE AMPylation reactions on separate microplates with our HTS buffer containing 0.1% TX-100 and 1 mM Mg2+ as a cofactor. Further, we maintained all other assay parameters as in our original pilot HTS, including enzyme concentration (400 nM), Fl-ATP concentration (25 nM), and reaction time (10 minutes). The low Fl-ATP concentration was specifically selected to not bias our screen away from compounds having an ATP-competitive mode of action; whereas the 10-minute time point ensured our reactions were within HYPE's linear range of AMPylation. Under these assay conditions, we observed excellent Z′ (i.e., 1>Z′≥0.5) and S/B values: 0.67 and 3.6, respectively. These were comparable to, yet lower than our pilot screen statistics (Z′=0.88 and S/B=4.6). These discrepancies could stem from our novel buffer conditions (i.e., TX-100 and Mg2+). Alternatively, the addition of DMSO (dimethyl sulfoxide) may have had a dampening effect on signal—particularly the E234G HYPE signal, as WT is already basal. Despite being the standard solvent for dissolving polar and nonpolar compounds found in chemical libraries, DMSO is not without its downsides. We and others have reported DMSO to impede with the enzymatic activities of multiple proteins (Camara et al. 2020; Tiernberg et al. 2006).

With a robust screening assay in hand, we next sought out an appropriate collection of compounds. In our pilot screen, we used a diverse set of compounds from four common HTS libraries, including Microsource Spectrum and LOAPC1280. While these libraries may be suitable for a pilot screen owing to the high probability of potential hits, their utility for discovering a HYPE-specific modulator is limited. This is because these bioactive compounds act against a broad range of cellular targets (Bisson et al. 2016). We, thus, selected from two libraries containing novel compounds which are less likely to have off-target effects (Table 1). The first of these libraries, DIVERset from Chembridge, was optimized for compound effectiveness by 3D conformational analysis, and stringent application of drug-like and functional group filters. The second library, CNS-Set also by Chembridge, uses a series of physicochemical filters (e.g., Lipinski Rules, Polar Surface Area, etc.) to enhance the probability of blood-brain barrier (BBB) penetration and bioavailability. Taken together, the more than 30,000 compounds from these two libraries ensures that potential hits will be amenable to downstream drug discovery efforts. The CNS-Set library was selected in anticipation of HYPE-centric treatments for neurodegeneration and neurogenesis (Sanyal et al. 2019; Truttmann et al. 2018; Kielkowski et al. 2020).

TABLE 1
Compound Library Information

| Library Name | Company | Number of Compounds | Chemical Properties |
|---|---|---|---|
| DIVERset | ChemBridge | 14,080 | Diverse |
| CNS-Set | ChemBridge | 16,000 | BBB penetrant |
| Total | — | 30,080 | — |

### WT HYPE Activator High-Throughput Screen

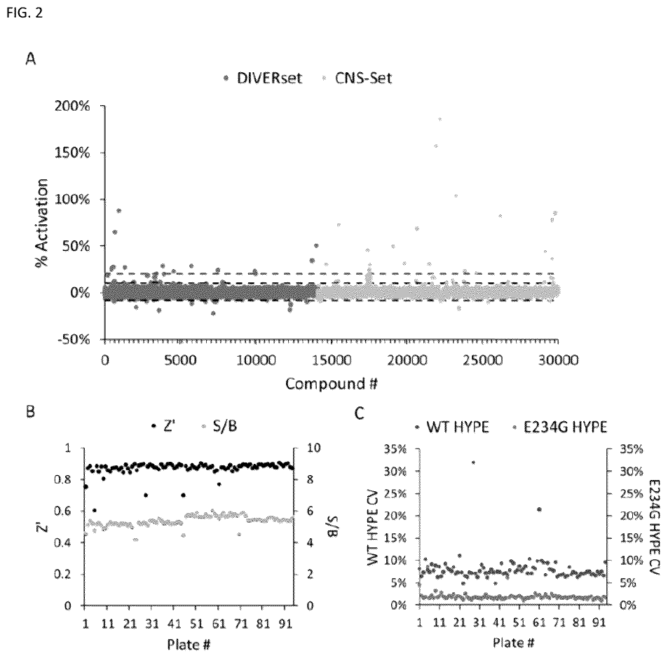

In the first half of our dual HTS, we challenged over 30,000 of these compounds against WT HYPE toward the discovery of novel AMPylation enhancers (FIG. 2A). Here, we define our hit threshold at 20% activation normalized to internal controls—WT HYPE with DMSO as a low control and E234G HYPE as a high control—which yields an overall hit rate of 0.12% activators (i.e., 36 hits out of 30,080 compounds). Among these 36 hits, 15 stemmed from the DIVERset library, while the other 21 were from the CNS-Set. To control for auto-fluorescing false positive compounds, or those intrinsically capable of quenching fluorescence emanating from Fl-ATP(/AMP), we manually filtered compounds containing outlier parallel intensities.

Excellent Z′ values and high S/B values were observed throughout our activator HTS, denoting a quality assay, with minimal plate-to-plate variability (FIG. 2B). Our coefficient of variance analysis also returned values consistently below the 10% limit, which defined an excellent assay. Notably, while all E234G HYPE high controls gave sub-10% CVs (most being sub-5%), the WT HYPE low controls displayed considerably more variability, with some falling outside of the sub-10% range. This difference likely results from the fact that at low, basal levels of WT HYPE AMPylation signal, slight variance in FP values get magnified relative to those of the much higher E234G signal. Nevertheless, we took care not to advance hits from plates having outlier quality statistics.

### WT HYPE Activator Microplate Validation

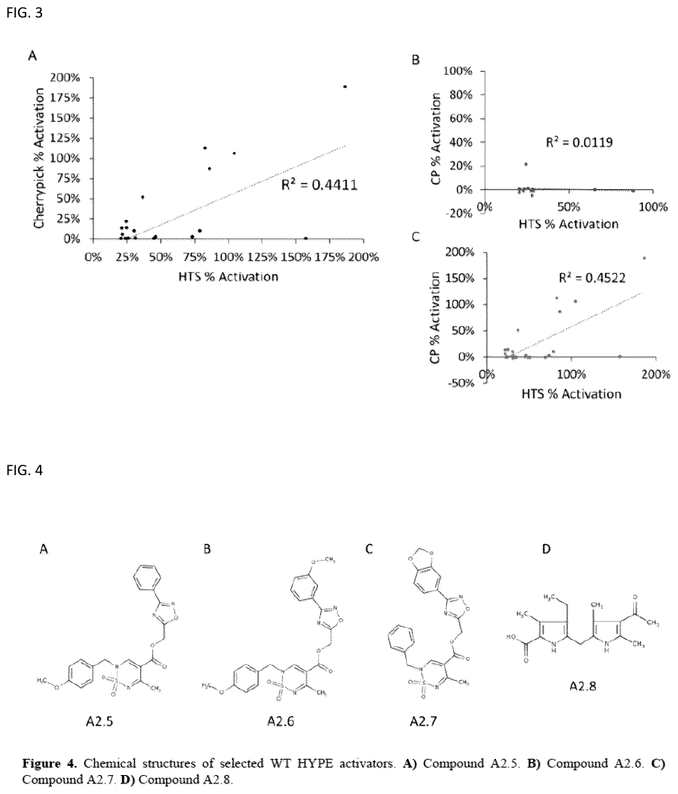

The hits from our activator HTS were cherry-picked for validation in an independent replication of the same HTS assay, likewise on microplate format. Upon second-pass selection, these hits yielded a modest R2 value of 0.44 with respect to the initial HTS (FIG. 3A). Interestingly, grouping hits based on their libraries of origin revealed even higher correlation among CNS-Set compounds (FIG. 3B), and radically lower correlation among DIVERset molecules. This could be explained by the higher initial bioactivities of CNS-Set compounds, thus reducing the likelihood of false positives.

We next advanced several hits for follow up validation using, chemical, diversity, biological activity, and commercial availability as selection criteria (FIG. 4A-D, Table 2). Of note, two of our hits—A2.6 and A2.7—had strikingly similar structures which differed only in the presence of an oxy-methyl group in A2.6. This suggests that these compounds may target HYPE at the same residues and with a similar mode of action. To control specifically for false positive compounds with intrinsically high fluorescence properties, we performed microplate FP AMPylation reactions with or without enzyme. We hypothesized that true positive hits would have low FP signal in the absence of HYPE and higher signal with HYPE; conversely false positives would maintain high “AMPylation” signal irrespective of an AMPylase. Indeed, we observed several false positive activators among our initially selected hits (FIG. S2A). After reselecting more hits from our reservoir of cherrypicked compounds, we were able to find four putative activators lacking intrinsic FP (FIG. S2B).

TABLE 2
WT HYPE Activator Hit Information

| | | | | | | % Act. | |
|---|---|---|---|---|---|---|---|
| Compound | Chemical | MW | Library | | % Act. | Cherry- | Mean % |
| ID | Formula | (Da.) | ID | Library | HTS | pick | Act. |
| A2.5 | C₂₂H₂₀N₄O₆S | 468 | S342- | CNS set | 30 | 10 | 20 |
| | | | 0409 | | | | |
| A2.6 | C₂₃H₂₂N₄O₇S | 499 | S342- | CNS set | 21 | 13 | 17 |
| | | | 0417 | | | | |
| A2.7 | C₂₂H₁₈N₄O₇S | 482 | S342- | CNS set | 25 | 14 | 19.5 |
| | | | 0376 | | | | |
| A2.8 | C₁₇H₂₂N₂O₃ | 302 | 2895- | CNS set | 37 | 52 | 44.5 |
| | | | 0212 | | | | |

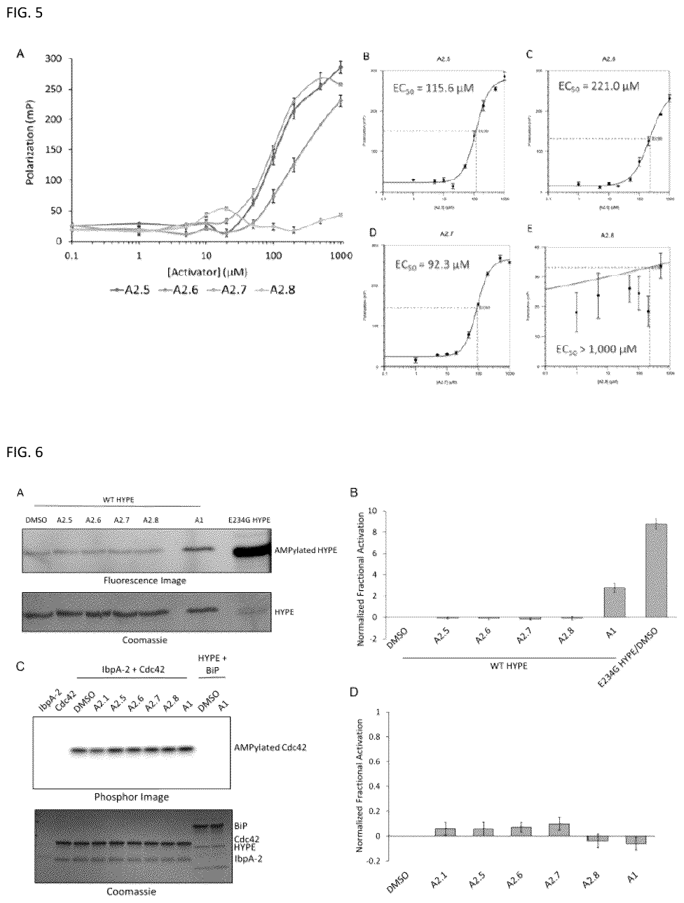

Concentration-response curves were conducted for these hits using increasing concentrations of our top putative activators under standard FP microplate reaction conditions (FIG. 5A-E). Three of our top four hits (A2.5-A2.7) display dose dependencies with EC50 values in the mid micromolar range. A2.8, however, has a poor response even at our highest compound concentration of 500 μM, which precludes EC50 quantification. This lack of strong concentration dependent response suggests A2.8 may be a false positive activator.

### WT HYPE Activator In-Gel Counterscreen

To further validate these activator hits, we employed orthogonal biochemical assays capable of directly tracking AMPylation in vitro. Besides monitoring FP, the Fl-ATP fluorophore is suitable for in-gel fluorescence AMPylation assays (Camara et al. 2020; Lewallen et al. 2015). Apart from the FP-based microplate assay—which indirectly correlates changes in AMPylation status to changes in fluorophore rotation—in-gel assays monitor the direct posttranslational modification of protein substrates separated by SDS-PAGE (sodium dodecyl sulfate polyacrylamide gel electrophoresis). After challenging our in-gel fluorescence AMPylation assay with hits from the FP microplate assays, we fail to see activation of AMPylation (FIG. 6A-B). Our top pilot screen hit, A1 (calmidazolium), did, however, prove bioactive above our DMSO control.

Given the relatively large size of the fluorescein ring conjugated to Fl-ATP, we wanted to control for possible interference with the AMPylation reaction. We, therefore, used a radiolabeled α-32P-ATP, which can monitor covalent attachment of AMP without the bulky fluorescent handle (Camara et al. 2020b). However, even with this more physiologically relevant nucleotide, no activation of AMPylation is apparent (data not shown). We previously reported differences in AMPylation signal sensitivity between an FP microplate format and in-gel assays, with the latter being less capable of detecting weaker signal (Camara et al. 2020). This is especially problematic for the hypoactive AMPylase WT HYPE. Unlike our hyperactive AMPylase E234G HYPE mutant, WT HYPE is a robust deAMPylase, which further compounds the issue of detecting of stable AMPylation (or manipulation thereof) (Ham et al. 2014; Preissler et al 2017). Moreover, our stringent selection filtering of putative hits (e.g., intrinsic parallel intensities, etc.), left us with less potent activators to begin with.

As a potential solution to detecting a weak WT HYPE AMPylation signal, we turned to evolution. HYPE is a member of the highly structurally and functionally conserved Fic domain protein family. One of the best studied Fic proteins, the bacterial effector IbpA, is known to AMPylate host cell Rho GTPases during its pathogenesis with high efficiency (Worby et al. 2009; Mattoo et al. 2011; Zekarias et al. 2010). To validate our activators independent of WT HYPE, we challenged to top putative activators against IbpA AMPylation of Cdc42 using radiolabeled α-32P-ATP (FIG. 6C-D). Since the IbpA toxin houses two separate Fic domains, we simplified our experimental design by targeting Cdc42 with a construct solely containing the second of the two domains, IbpA-2. Consistent with our WT HYPE data, none of the putative activators increased IbpA-2's AMPylation of Cdc42 beyond our DMSO controls. The lack of AMPylation enhancement by our top pilot screen WT HYPE activator, A1, is also consistent with previous results (Camara et al. 2020).

### E234G HYPE High-Throughput Screen

Under the same reaction parameters as our WT HYPE activator HTS, we re-screened the 30,080 compounds from DIVERset and CNS-Set libraries (Table 1) for inhibition of E234G HYPE. Using the same 20% hit definition, we saw an overall hit rate of 0.38% (i.e., 114 hits out of 30,080 compounds), more than three times that of our activator screen. Given the ease with which catalysis can be disrupted versus enhanced, this relatively higher rate of inhibitor hits is as expected (Saboury et al. 2009). Among these 114 hits, 32 stemmed from the DIVERset library, while 82 were from the CNS-Set. To control for auto-fluorescing false positive compounds, or those intrinsically capable of quenching fluorescence emanating from Fl-ATP(/AMP), we manually filtered compounds containing outlier parallel intensities.

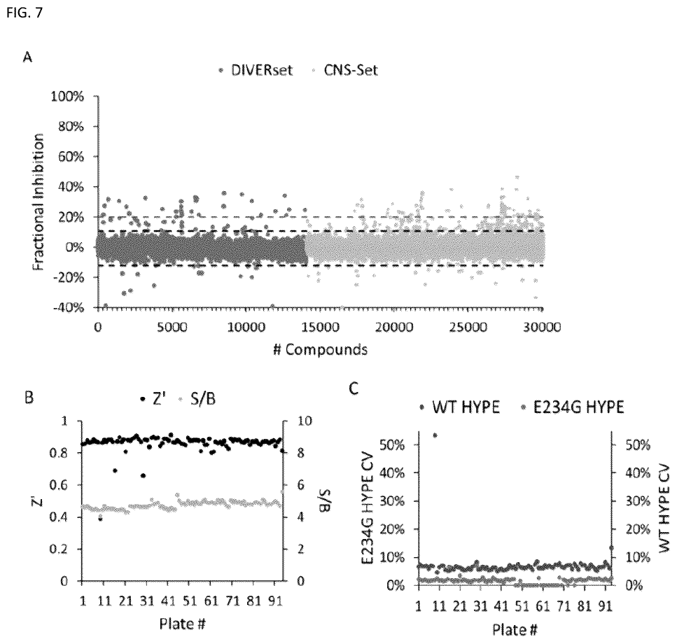

As in our activator screen, here we maintained excellent Z′ values and high S/B values throughout our inhibitor HTS, denoting a quality assay, with minimal plate-to-plate variability (FIG. 7B). We did, however, have a single plate (FIG. 7B, plate #9) with an unacceptably low Z′ value. Since no 20% hits happened to be on this plate, this anomaly was inconsequential to our overall screening process. We also observed coefficient of variance values consistently below the 10% limit defining an excellent assay, with only one exception for WT HYPE plate #9 (FIG. 7C). As in our activator HTS, all E234G HYPE high controls gave sub-5% CVs, while the WT HYPE low controls yielded higher values with variability. The reason for this increased variance is likely the same (i.e., low, basal levels of WT HYPE AMPylation signal amplify minor variance in FP values relative to those of the much higher E234G signal).

### E234G HYPE Inhibitor Microplate Validation

Hits from our inhibitor screen were then cherry-picked in an independent replication of the same HTS microplate assay. Strikingly, little correlation was observed from the initial HTS to the cherrypick validation (FIG. 8A), although this correlation was slightly improved for the DIVERset library (FIG. 8B-C). These all-around poor correlations could in part result from the low hit definition of 20%, as a more stringent threshold leads to increased reproducibility (Camara et al. 2020).

TABLE 3
E234G HYPE Inhibitor Hit Information

| | | | | | | % Inh. | |
|---|---|---|---|---|---|---|---|
| | Chemical | MW | Library | | % Inh. | Cherry- | Mean % |
| Compound | Formula | (Da.) | ID | Library | HTS | pick | Inh. |
| I2.1 | C_{QR}H_{HN}N_{L} | 298.4 | 74731416 | DIVERset | 32.5 | 29.7 | 31.1 |
| I2.2 | C_{Q} [symbol] H_{QN}N_{H}O [symbol] | 300.3 | 88812993 | DIVERset | 20.3 | 22.7 | 21.5 |
| I2.3 | C_{HQ}H_{Q} [symbol] N_{S}O_{H} | 371.4 | 57920321 | DIVERset | 35.0 | 30.7 | 32.9 |
| I2.4 | C_{Q} [symbol] H [symbol] _{L}N_{L}O | 242.3 | 8561-07647 | CNS-Set | 27.8 | 31.0 | 29.4 |
| I2.5 | C_{H} [symbol] H_{QS}F_{H}N_{S}O | 379.4 | J094-0169 | CNS-Set | 23.5 | 32.9 | 28.2 |
| I2.6 | C₂₀H₁₅BrFN₅O | 440.3 | J094-0175 | CNS-Set | 32.7 | 29.7 | 31.2 |
| I2.7 | C₁₅H [symbol] BrN [symbol] OS | 360.2 | 5089-0088 | CNS-Set | 20.9 | 34.2 | 27.6 |
| I2.8 | C [symbol] H₂₀N₆O₂ | 352.4 | 80384857 | CNS-Set | 25.2 | 29.1 | 27.2 |
| I2.9 | C₂₁H [symbol] N₄O₂ | 358.4 | S343-0959 | CNS-Set | 27.2 | 27.8 | 27.5 |
| I2.10 | C₁₄H₉N₅O₂ | 279.3 | P901-0269 | CNS-Set | 24.5 | 29.7 | 27.1 |
| I2.11 | C₁₅H [symbol] N₂O | 242.3 | D014-0004 | CNS-Set | 19.9 | 24.6 | 22.3 |
| I2.12 | C₁₆H₁₁FN₄O | 294.3 | 8561-07646 | CNS-Set | 27.1 | 33.6 | 30.4 |

[symbol]

indicates data missing or illegible when filed

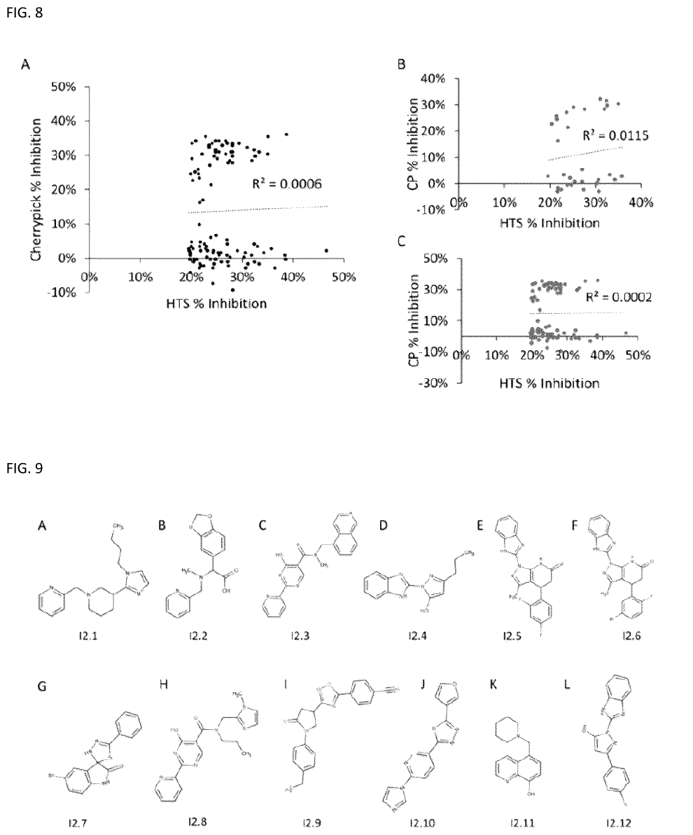

Using chemical diversity, biological activity, and commercial availability as selection criteria, we forward processed our top hits for validation (FIG. 9A-L, Table 3). Inspectional analysis of these compounds' chemical structures revealed several motifs, including a two ringed, purine-like structure seen in I2.4-I2.7, and I2.12 (FIG. 9D-G, 9L). This feature is structurally reminiscent of ATP's nitrogenous base. Moreover, these results are consistent with our pilot screen, in which we identified several ATP analogs as top inhibitor hits (Camara et al. 2020). Global structural motifs can be found amongst other putative hits: I2.5 and I2.6 (FIG. 9E-F), by example, differ only in the position and variety of alkyl halides on their terminal benzene ring. Taken together, these data hint at similar mechanisms of action among the hits.

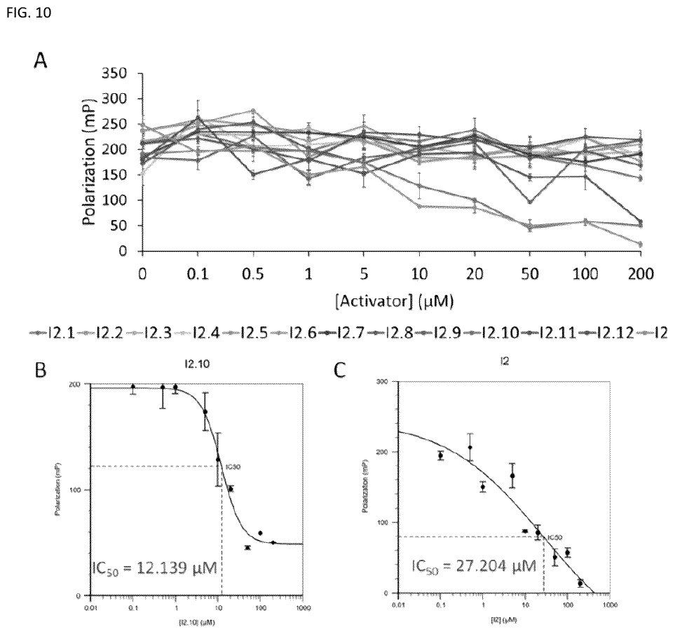

We next performed concentration-response curves for these hits using increasing concentrations of our top putative inhibitors under standard FP microplate reaction conditions (FIG. 10A). Though most putative inhibitors failed to display robust dose dependent inhibition of AMPylation, I2.10 showed high potency, with low micromolar IC50 values (FIG. 10A). Indeed, I2.10's bioactivity was on par with that of our top validated pilot screen hit I2 (aurintricarboxylic acid; FIG. 10C). Unlike I2, however, I2.10 is a novel chemical with no known biological targets, which holds promise for potential drug candidacy.

### E234G HYPE Inhibitor In-Gel Counterscreen

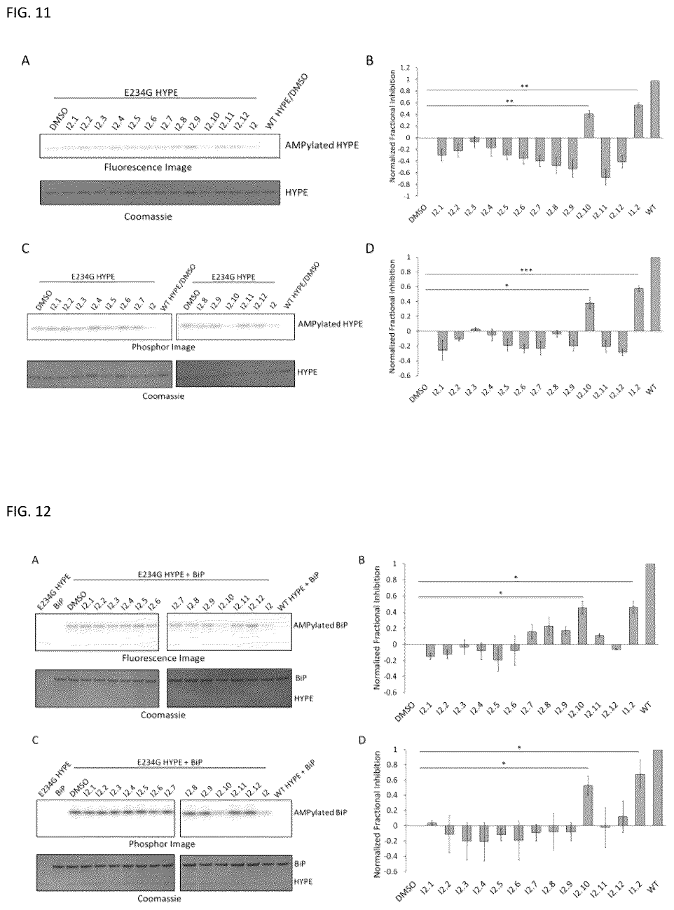

Putative inhibitors were then subjected to similar in-gel fluorescence and radiolabeled validations as our activators to directly probe for protein AMPylation. Starting with our fluorescent in-gel assay, we see that I2.10 consistently perturbs HYPE autoAMPylation in vitro (FIG. 11A-B), using Fl-ATP as a nucleotide source. The bioactivity of I2.10 is mirrored under the more physiological conditions of AMPylation with an α-32P-ATP substrate (which lacks the bulky fluorophore; FIG. 11C-D).

Despite an ever-expanding AMPylome, the ER heat shock chaperone BiP remains HYPE's only fully validated protein substrate to date. We, therefore, assessed the ability of our novel top inhibitor, I2.10, to manipulated HYPE-promoted AMPylation of BiP. Intracellularly, BiP exists in two conformation states, and fluctuates between these depending on its position in the chaperone cycle. During homeostasis, BiP adopts an open conformation which favors AMPylation and disfavors BiP binding to its unfolded protein substrates. Alternatively, triggering of the UPR under stress conditions leads to deAMPylation of BiP, thus permitting it to bind to and fold its misfolded protein clients in a closed conformation. Given this chaperone cycle dependent conformational preference, and the fact that WT BiP is thought to dynamically fluctuate between conformations, we opted to perform our in vitro AMPylation assays with a mutant BiP construct that's constitutively locked in an open, AMPylation-competent state. Using this T229A BiP, and Fl-ATP as a co-substrate, we were able to confirm the inhibitory properties of I2.10 (FIG. 12A-B). Further, I2.10's inhibitory efficacy against BiP AMPylation was reproducible using the more physiological α-32P-ATP (FIG. 12C-D). As with our autoAMPylation results, I2.10 inhibited BiP AMPylation comparable to the I2 positive control.

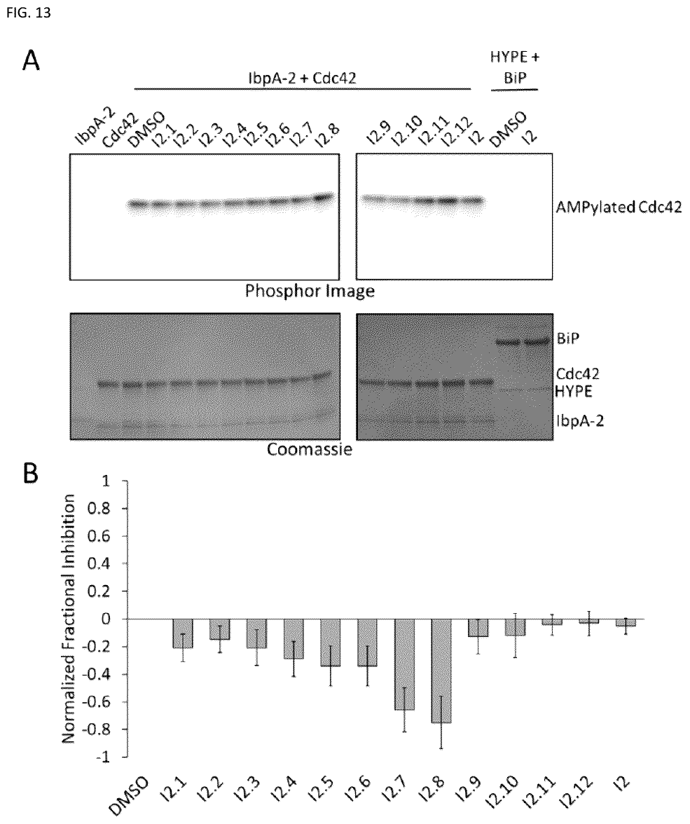

To assess the cross reactivity of our putative inhibitors, we conducted in vitro AMPylation assays using IbpA-2 and Cdc42 with radiolabeled α-32P-ATP. We observed no manipulation of AMPylation under these conditions (FIG. 13). Consistent with our pilot screen, the I2 control also had no impact on AMPylation of Cdc42. This may be due to sequence and/or structural differences between the Fic domains of HYPE and IbpA-2. In fact, comparative structural analyses of the HYPE:BiP and IbpA:Cdc42 co-crystal structures reveal only partial superimposition among their respective Fic domain active sites (Fauser et al. 2021).

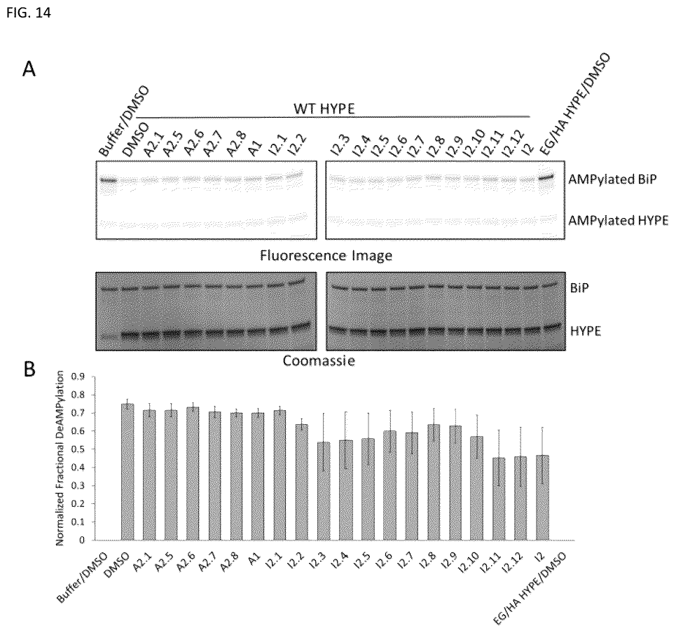

E234G HYPE, though a hyperactive AMPylase, is deAMPylation deficient owing to the lack of formation of a supportive salt bridge between E234 and R374. Moreover, this regulatory glutamate is essential in deAMPylation, as it coordinates a catalytic water molecule within the Fic active site to nucleophilically attack the phosphodiester bond joining AMP to the target residue. WT HYPE, with its intact E234, is thus a robust deAMPylase. Given the HYPE's role in deAMPylation-mediated activation of BiP's chaperoning activity during the response to cellular stress (i.e., UPR induction), we wanted to determine our hit compounds' ability to manipulate deAMPylation. We hypothesized that since AMPylation and deAMPylation occur at the same Fic catalytic pocket—albeit by slightly different mechanisms—compounds affecting HYPE's AMPylase activity could likewise modulate the reversing post-translational modification. To test this, we conducted a standard in vitro two-step deAMPylation assay: 1) E234G HYPE and Fl-ATP is used to AMPylate T229A BiP to completion, followed by incubation with deAMPylation competent WT HYPE is the presence or absence of compound (FIG. 14A). Under these conditions, we witnessed no enhancement or inhibition by our hit compounds of the current or pilot HTS. Intriguingly, we did see a lower deAMPylation signal in most of the inhibitors than in the activators or DMSO controls; however, none of these differences were statistically significant (FIG. 14B).

### Cellular Toxicity Validation

Before a hit compound can be advance to drug candidacy, it must prove nontoxic to its target cells. We, therefore, determined the cellular toxicity of our top hit, I2.10, using a standard MTT cell viability assay with standard in vitro cell line models: HeLa and HEK 293. MTT is a yellow dye that gets reduced to purple formazan crystals in presence of NADPH proton donors29. Since abundant NADPH production is a hallmark of cellular respiration, we're able to correlate cell viability to generation of purple crystals by measuring the absorbance at 570 nm.

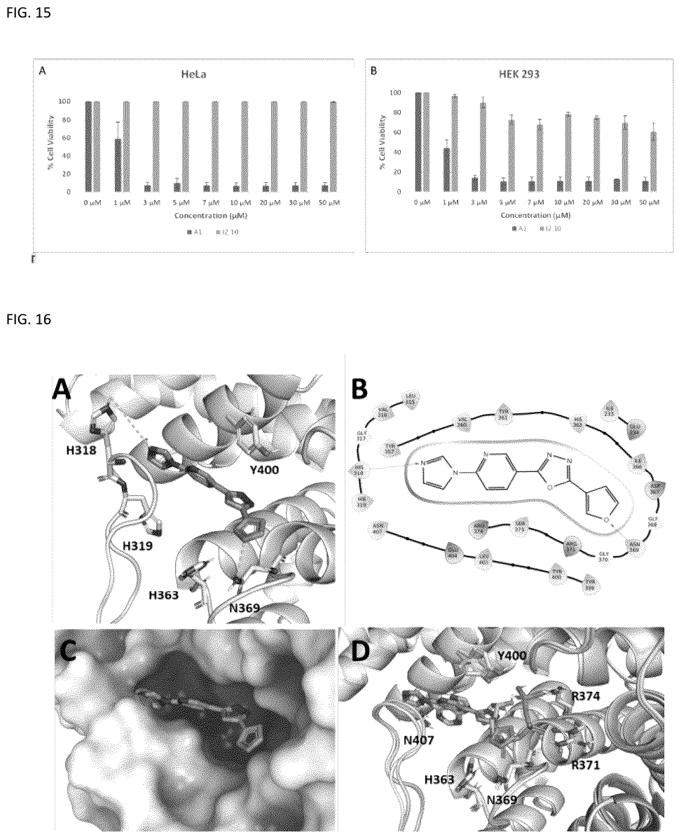

Given I2.10's robust bioactivity at 10 μM, we wanted to assess whether this concentration would be suitable for cellular treatment. Our previous top activator, A1, was used a control due to its reported cellular toxicity. After incubating HeLa cells in varying amounts of compound, we observed no toxicity (100% cell viability) from I2.10 (FIG. 15A), even at concentrations five times higher than its in vitro IC50 (FIG. 10A-B). Notably, our maximum experimental concentration of 50 μM corresponds to the in vitro saturation point of I2.10-mediated AMPylation inhibition (i.e., zero percent AMPylation). This is a promising finding and suggests the potential for total chemical ablation of cellular AMPylation with no deleterious effects to the health of the target cell.

As expected, our A1 control impacted cell viability with as little as 1 μM (FIG. 15A). Due to the promiscuous nature of this compound, we cannot unequivocally deduce the mechanisms of cytotoxicity (Gietzen et al. 1981; Sunagawa et al. 2000; James et al. 2009). However, it is plausible that A1's reported activation of AMPylation is at play. This result has been genetically phenocopied by cellular transfection with the hyperactive E234G HYPE, which is known to induce caspase-dependent apoptosis.

The relative toxicity trends seen in HeLa cells remained consistent in HEK 293. I2.10 treated HEK 293 cells retain ˜80% cell viability at our bioactive concentration of 10 μM (FIG. 15B). However, we did observe somewhat of a concentration-dependent effect in I2.10's toxicity in HeLa, dropping to ˜60% cell viability at 50 μM. Though further investigation is needed to elucidate this cell specificity in I2.10-induced toxicity, it's possible that these arise from inherent biochemical differences between HeLa and HEK 293 cells, potentially providing novel targets for I2.10.

### Docking Studies

To better understand how I2.10 may be interacting with HYPE a computational approach was taken via docking studies. The molecule was docked using the Glide Extra Precision (XP) method that allows for flexible ligand and receptor docking. The apo-HYPE structure (PDB: 4U04) was prepared using the Protein Preparation Wizard available through the Maestro interface of the Schrodinger platform, and I2.10 was prepared using LigPrep. Next, the receptor grid was generated at the ATP-binding site defined by Fic moiety residues His363, Arg371, Arg374, and Tyr400. Then I2.10 was docked into the grid.

The top pose for I2.10 was selected for analysis by considering docking score and protein-ligand interactions with residues in the ATP-binding site. Most noticeably, I2.10 fits within the ATP-binding site pocket and interacts with residues within this site (FIG. 16). Notably, I2.10 is mostly planar and hydrophobic in nature leading to the predicted pose forming extensive hydrophobic interactions with aromatic residues within the ATP-binding pocket (FIG. 16). These hydrophobic interactions are flanked by two accepted hydrogen bonds, one between the imidazole accepting an H-bond from His318 and the other between the furan oxygen accepting an H-bond from the side chain of Asn369. Interestingly, in the ATP-bound structure (PDB: 4U07), the aromatic adenine moiety participates in ρ-ρ interactions with Tyr400. The docked pose for I2.10 overlaid with the observed ATP-bound conformation (FIG. 16D) shows the pyridine heterocycle from I2.10 aligning well with the adenine moiety from ATP and is in proximity with to Tyr400. Thus, it is feasible based on this docking study that I2.10 binds to the ATP-site and gains two hydrogen bonds but also maintains a similar interaction with Tyr400. While these docking studies are promising for elucidating how I2.10 may inhibit HYPE further work is required to fully characterize the binding mode and validate these docking results. Nonetheless, the docked pose is informational for the design of these studies.

HYPE's regulatory position at the nexus of various disease pathways makes it an attractive therapeutic target. However, to date, no novel compounds directed against HYPE-mediated AMPylation have been discovered. Here, we establish an optimized and scalable dual screening assay capable of identifying novel modulators of in vitro HYPE AMPylation. We also present a robust and reproducible pipeline for validating hits using orthogonal biochemical and cellular assays. Indeed, we've discovered a novel inhibitor of HYPE-directed AMPylation in I2.10. We report consistent I2.10-induced reduction of HYPE autoAMPylation with low micromolar bioactivity. Moreover, I2.10 enacts a reliable inhibitory effect of AMPylation on HYPE's primary substrate, the ER chaperone BiP. The high bioactivity and low cytotoxicity of I2.10 suggests it could act specifically against cellular targets—both BiP and other emerging substrates such as alpha synuclein. As it already possesses CNS amenable characteristics (e.g. BBB penetrant), I2.10 is especially suited for development as a HYPE-specific drug towards the treatment of alpha synuclein implicated Parkinson's disease and other neurodegenerative conditions. Ongoing follow-up experiments will determine the cellular efficacy of I2.10.

Docking studies predict that I2.10 binds in the ATP-binding site by accepting two hydrogen bonds and interacting with residues known to interact with the adenine functionality on ATP. Further validation of binding pose will inform medicinal chemistry efforts to generate I2.10 derivatives with enhanced drug likeness and bioactivity. I2.10's relatively low molecular weight (Table 3) and log P values make it well positioned for hit-to-lead optimizations.

### REFERENCES

1. Yarbrough, M. et al. AMPylation of Rho GTPases by Vibrio VopS Disrupts Effector Binding and Downstream Signaling. Science (80-. ). 323, 269-272 (2009).
2. Worby, C. A. et al. The Fic domain: A new paradigm for adenylylation. Mol. Cell 34, 1-23 (2009).
3. Ham, H. et al. Unfolded protein response-regulated Drosophila Fic (dFic) protein reversibly AMPylates BiP chaperone during endoplasmic reticulum homeostasis. J. Biol. Chem. 289, 36059-36069 (2014).
4. Sanyal, A. et al. A novel link between fic (filamentation induced by cAMP)-mediated adenylylation/AMPylation and the unfolded protein response. J. Biol. Chem. 290, 8482-8499 (2015).
5. Preissler, S., Rato, C., Perera, L., Saudek, V. & Ron, D. FICD acts bi-functionally to AMPylate and de-AMPylate the endoplasmic reticulum chaperone BiP. Nat Struct Mol Biol. 24, 23-29 (2017).
6. Sanyal, A. et al. Alpha-Synuclein Is a Target of Fic-Mediated Adenylylation/AMPylation: Possible Implications for Parkinson's Disease. J. Mol. Biol. 431, (2019).
7. Truttmann, M. C., Pincus, D. & Ploegh, H. L. Chaperone AMPylation modulates aggregation and toxicity of neurodegenerative disease-associated polypeptides. Proc. Natl. Acad. Sci. U.S.A. 115, E5008-E5017 (2018).
8. Kielkowski, P. et al. FICD activity and AMPylation remodelling modulate human neurogenesis. Nat. Commun. 11, 1-13 (2020).
9. Camara, A. et al. A fluorescence polarization-based high-throughput screen to identify the first small-molecule modulators of the human adenylyltransferase hype/ficd. Int. J. Mol. Sci. 21, 1-20 (2020).
10. Lewallen, D. M. et al. Inhibiting AMPylation: A novel screen to identify the first small molecule inhibitors of protein AMpylation. ACS Chem. Biol. 9, 433-442 (2015).
11. Gietzen, K., Wurthrich, A. & Bader, H. R 24571: A New Powerful Inhibitor of Red Blood Cell Ca2++-Transport ATPase and of Calmodulin-Regulated Functioons. Biochem. Biophys. Res. Commun. 101, 418-425 (1981).
12. Sunagawa, M., Kosugi, T., Nakamura, M. & Sperelakis, N. Pharmacological actions of calmidazolium, a calmodulin antagonist, in cardiovascular system. Cardiovasc. Drug Rev. 18, 211-221 (2000).
13. James, L. R., Griffiths, C. H., Garthwaite, J. & Bellamy, T. C. Inhibition of nitric oxide-activated guanylyl cyclase by calmodulin antagonists. Br. J. Pharmacol. 158, 1454-1464 (2009).
14. McCune, S. A., Foe, L. G., Kemp, R. G. & Jurin, R. R. Aurintricarboxylic acid is a potent inhibitor of phosphofructokinase. Biochem. J. 259, 925-927 (1989).
15. Hashem, A. M. et al. Aurintricarboxylic acid is a potent inhibitor of influenza A and B virus neuraminidases. PLoS One 4, 14-16 (2009).
16. Obrecht, A. S. et al. Identification of aurintricarboxylic acid as a potent allosteric antagonist of P2X1 and P2X3 receptors. Neuropharmacology 158, (2019).
17. Coan, K. E. D., Maltby, D. A., Burlingame, A. L. & Shoichet, B. K. Promiscuous aggregate-based inhibitors promote enzyme unfolding. J. Med. Chem. 52, 2067-2075 (2009).
18. Mattoo, S. et al. Comparative analysis of Histophilus somni immunoglobulin-binding protein A (IbpA) with other Fic domain-containing enzymes reveals differences in substrate and nucleotide specificities. J. Biol. Chem. 286, 32834-32842 (2011).
19. Camara, Ali; Sanyal, Anwesha; Dutta, Sayan; Rochet, Jean-Christophe; Mattoo, S. In vitro AMPylation/Adenylylation of Alpha-synuclein by HYPE/FICD. Bio-Protocol (2020).
20. Engel, P., Goepfert, A. & Dehio, C. Adenylylation control by intra- or intermolecular active-site obstruction in Fic proteins. Nature 482, 107-112 (2012).
21. Lewallen, D. M., Steckler, C. J., Knuckley, B., Chalmers, M. J. & Thompson, P. R. Probing adenylation: Using a fluorescently labelled ATP probe to directly label and immunoprecipitate VopS substrates. Mol. Biosyst. 8, 1701-1706 (2012).
22. Perera, L. A. et al. Structures of a deAMPylation complex rationalise the switch between antagonistic catalytic activities of FICD. Nat. Commun. 12, 1-18 (2021).
23. Fauser, J. et al. Specificity of AMPylation of the human chaperone BiP is mediated by TPR motifs of FICD. Nat. Commun. 12, (2021).
24. Zhang, Ji-Hu; Chung, Thomas D.; Oldenburg, K. R. A Simple Statistical Parameter for Use in Evaluation and validation of High Throughput Screening Assays. J. Biomol. Screen. 4, 67-73 (1999).
25. Tjernberg, A., Markova, N., Griffiths, W. J. & Hallen, D. DMSO-related effects in protein characterization. J. Biomol. Screen. 11, 131-137 (2006).
26. Bisson, J. et al. Can Invalid Bioactives Undermine Natural Product-Based Drug Discovery? J. Med. Chem. 59, 1671-1690 (2016).
27. Zekarias, B. et al. Histophilus somni IbpA DR2/Fic in virulence and immunoprotection at the natural host alveolar epithelial barrier. Infect. Immun. 78, 1850-1858 (2010).
28. Saboury, A. A. Enzyme inhibition and activation: A general theory. J. Iran. Chem. Soc. 6, 219-229 (2009).
29. Van Meerloo, J., Kaspers, G. J. L. & Cloos, J. Cell sensitivity assays: The MTT assay. Methods Mol. Biol. 731, 237-245 (2011).

Those skilled in the art will recognize that numerous modifications can be made to the specific implementations described above. The implementations should not be limited to the particular limitations described. Other implementations may be possible.

While the disclosure contained herein have been illustrated and described in detail in the drawings and foregoing description, the same is to be considered as illustrative and not restrictive in character, it being understood that only certain embodiments have been shown and described and that all changes and modifications that come within the spirit of the disclosure, are desired to be protected.

It is intended that the scope of the present methods and apparatuses be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

1. A pharmaceutical formulation comprising a compound having a formula (I) 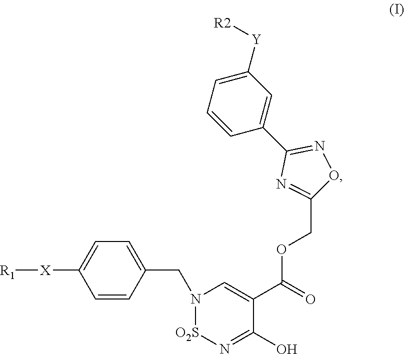
or a pharmaceutically acceptable salt thereof, wherein
X is H or O;
Y is H or O;
R₁ is a C₁-C₁₂ alkyl;
R₂ is a C₁-C₁₂ alkyl; heteroalkyl, heteroalkenyl, heteroalkynyl, cycloalkyl, heterocyloalkyl, cycloalkenyl, heterocycloakenyl, heterocyclyl, or cycloalkene,
and a pharmaceutically acceptable carrier, excipient or diluent.

2. The formulation of claim 1 wherein the compound is selected from the group consisting of:

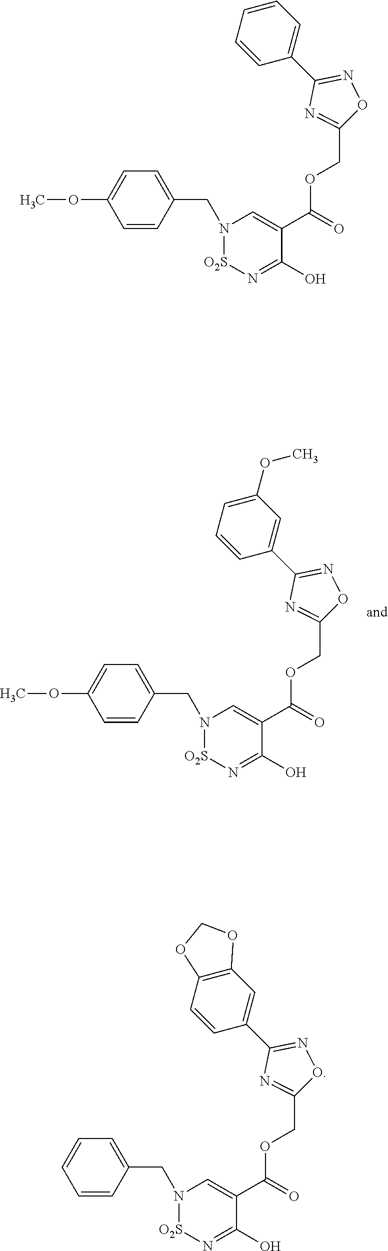 [SMILES: COc1ccc(CN2C=C(C(=O)OCc3nc(-c4ccccc4)no3)C(O)=NS2(=O)=O)cc1 ; COc1ccc(CN2C=C(C(=O)OCc3nc(-c4cccc(OC)c4)no3)C(O)=NS2(=O)=O)cc1 ; O=C(OCc1nc(-c2ccc3c(c2)OCO3)no1)C1=CN(Cc2ccccc2)S(=O)(=O)N=C1O]

3. A pharmaceutical composition comprising a compound having a formula (II) 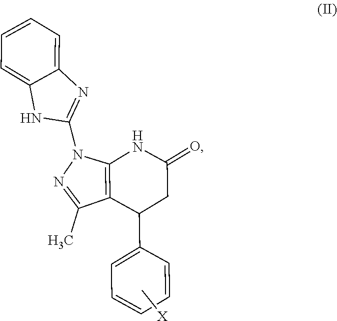
or a pharmaceutically acceptable salt thereof, wherein
X is 1 or 2 optionally bound halo-substituent to the ring structure,
and a pharmaceutically acceptable carrier, excipient or diluent.

4. The formulation of claim 3 selected from the group consisting of

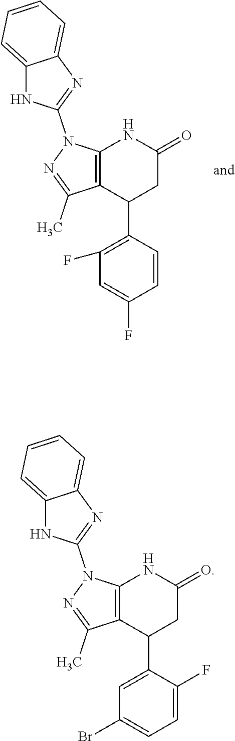 [SMILES: Cc1nn(-c2nc3ccccc3[nH]2)c2c1C(c1ccc(F)cc1F)CC(=O)N2 ; Cc1nn(-c2nc3ccccc3[nH]2)c2c1C(c1cc(Br)ccc1F)CC(=O)N2]

5. A pharmaceutical formulation comprising a compound having a formula (III) 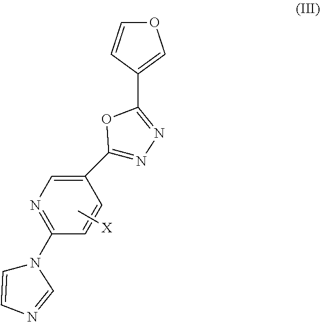
or a pharmaceutically acceptable salt thereof, wherein
X is 1 or 2 optionally bound halo-substituent to the ring structure,
and a pharmaceutically acceptable carrier, excipient or diluent.

6. A method for treating a patient for HYPE-mediated disease comprising administering a therapeutically effective amount of the formulation of claim 1 to the patient in need of relief from said disease.

7. A method for treating a patient for HYPE-mediated disease comprising administering a therapeutically effective amount of the formulation of claim 3 to the patient in need of relief from said disease.

8. A method for treating a patient for HYPE-mediated disease comprising administering a therapeutically effective amount of the formulation of claim 5 to the patient in need of relief from said disease.

9. A compound selected from the group consisting of:

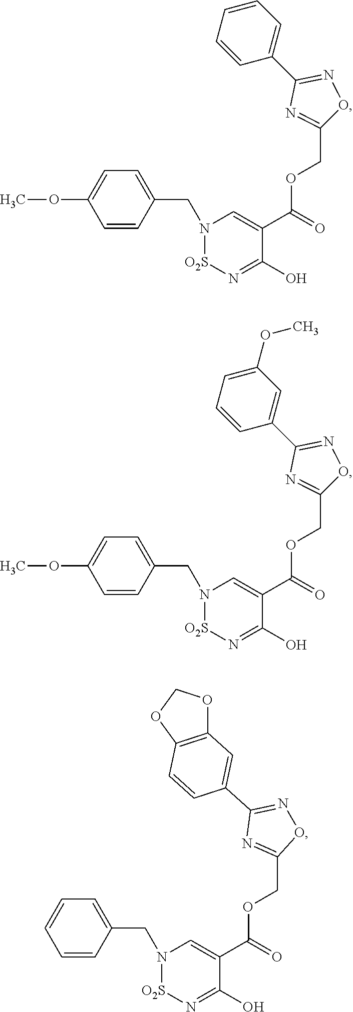 [SMILES: COc1ccc(CN2C=C(C(=O)OCc3nc(-c4ccccc4)no3)C(O)=NS2(=O)=O)cc1 ; COc1ccc(CN2C=C(C(=O)OCc3nc(-c4cccc(OC)c4)no3)C(O)=NS2(=O)=O)cc1 ; O=C(OCc1nc(-c2ccc3c(c2)OCO3)no1)C1=CN(Cc2ccccc2)S(=O)(=O)N=C1O]

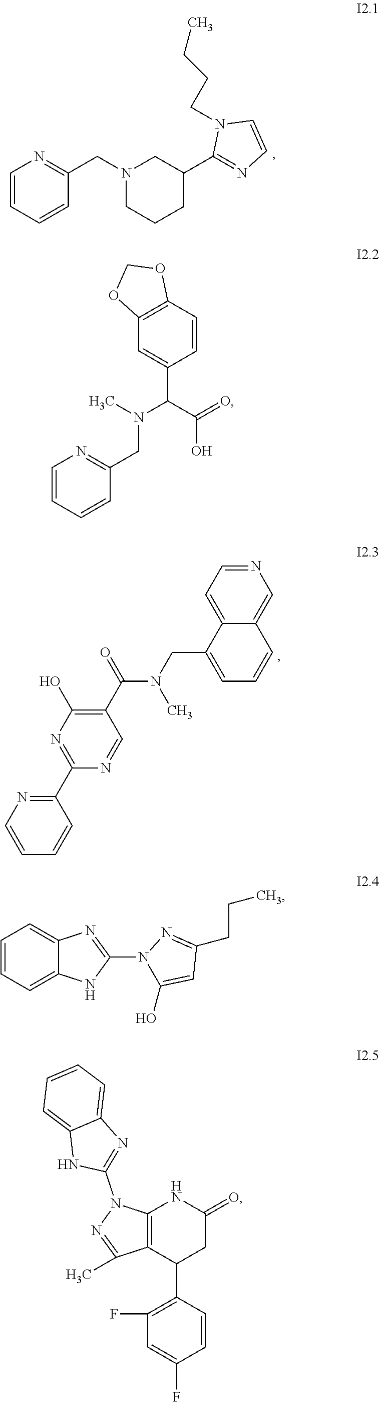

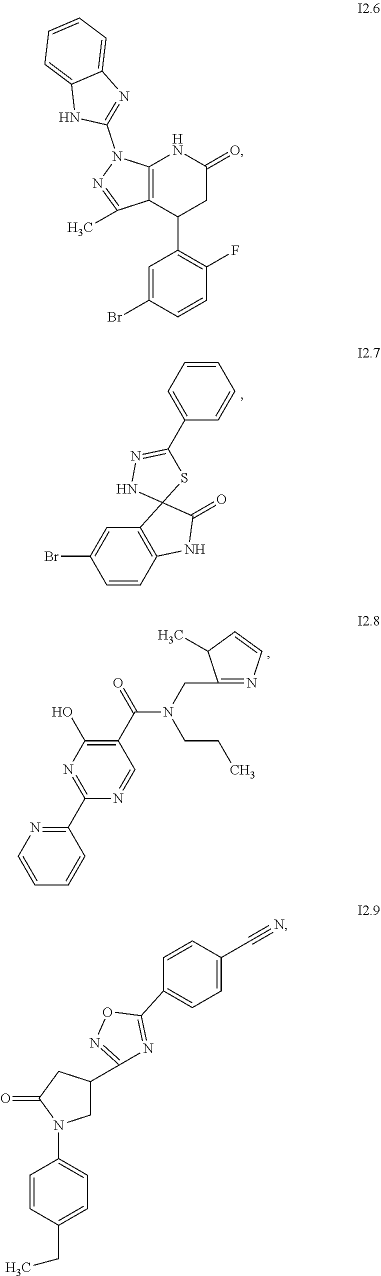

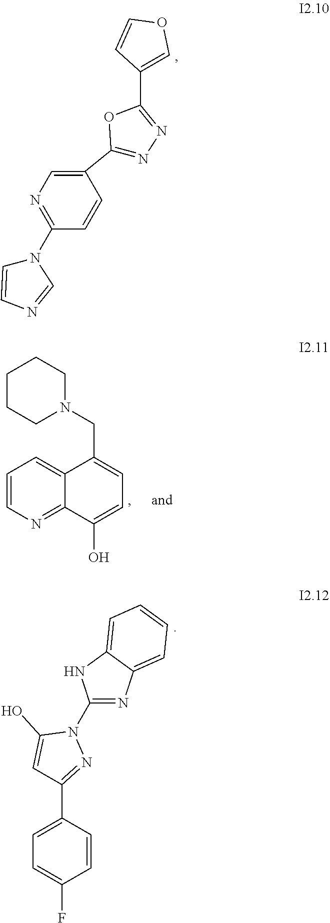

10. A compound or claim 9 having the formula selected from the group consisting of: 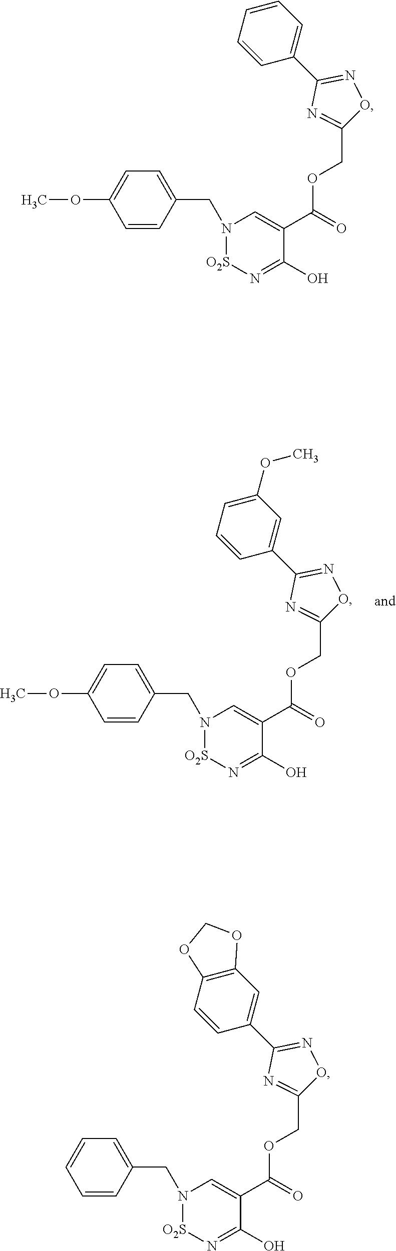 [SMILES: COc1ccc(CN2C=C(C(=O)OCc3nc(-c4ccccc4)no3)C(O)=NS2(=O)=O)cc1 ; COc1ccc(CN2C=C(C(=O)OCc3nc(-c4cccc(OC)c4)no3)C(O)=NS2(=O)=O)cc1 ; O=C(OCc1nc(-c2ccc3c(c2)OCO3)no1)C1=CN(Cc2ccccc2)S(=O)(=O)N=C1O]
which is an agonist of HYPE-mediated AMPylation.

11. A compound or claim 9 having the formula selected from the group consisting of: 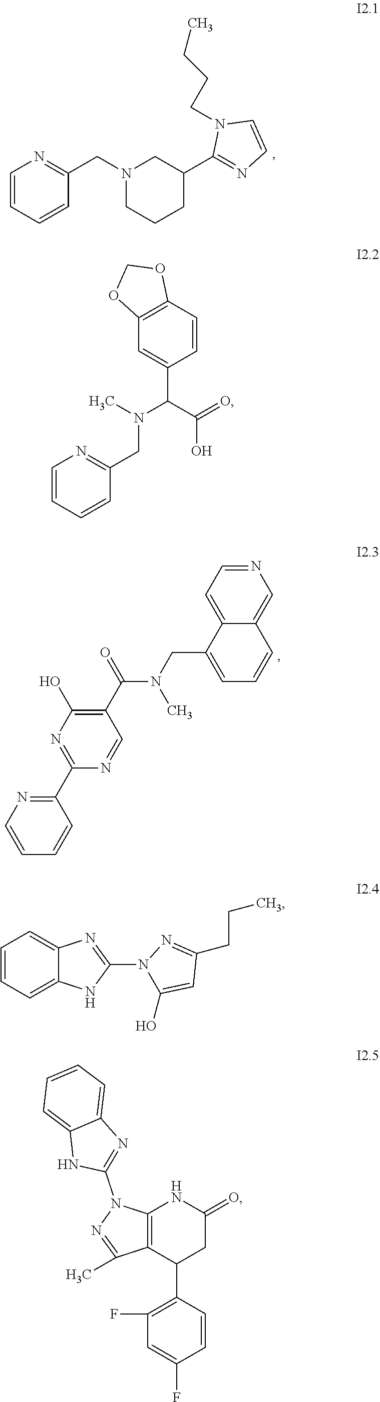 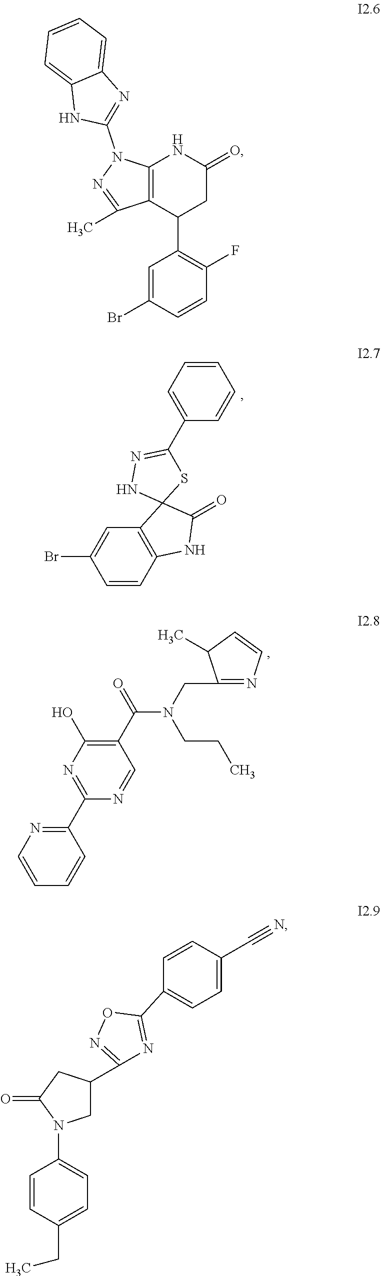 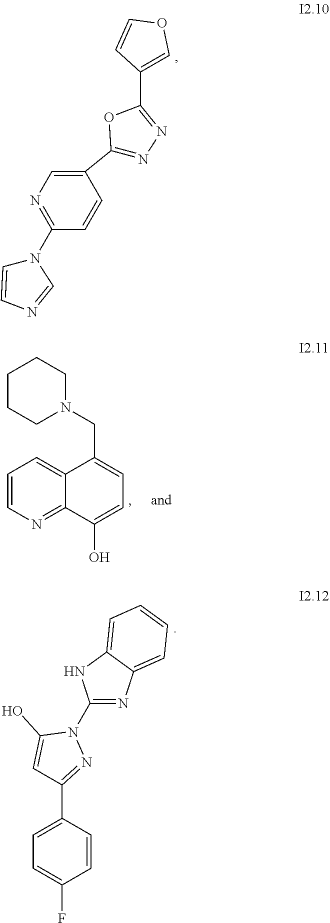
which is an inhibitor of HYPE-mediated AMPylation.

12. A pharmaceutical composition comprising a therapeutically effective amount of a compound of claim 9 and a pharmaceutically acceptable carrier, excipient or diluent.

13. A method for treating a patient for HYPE-mediated disease comprising administering a therapeutically effective amount of the compound of claim 9 to the patient in need of relief from said disease.

14. A method for treating a patient for HYPE-mediated neurodegenerative disease comprising administering a therapeutically effective amount of the compound of claim 9 to the patient in need of relief from said disease.

15. A method for screening for an activator of HYPE-mediated AMPylation comprising preparing a test environment having a modified AMPylase HYPE in a Mg buffer, contacting a test small molecule with the test environment, and detecting signal, where said method is amenable to tracking HYPE's enzymatic activity at scale with automation.

16. The method of claim 15 using hypoactive AMPylase WT HYPE and Mg Buffer to screen for potential specific activators.

17. The method of claim 15 using hyperactive E234G HYPE and Mg Buffer to screen for potential specific inhibitors.
